(19) United States
(12) Patent Application Publication
(10) Pub. No.: US 2024/0358938 A1
(43) Pub. Date: Oct. 31, 2024

(54) INSUFFLATOR AND AN ENDOSCOPE, AN INSUFFLATING SYSTEM AND A METHOD FOR CARRYING OUT A PROCEDURE COMPRISING AN INSUFFLATOR AND AN ENDOSCOPE

(71) Applicant: Palliare Limited, Dangan (IE)

(72) Inventor: John O’DEA, Galway (IE)

(73) Assignee: Palliare Limited, Dangan (IE)

(21) Appl. No.: 18/658,042

(22) Filed: May 8, 2024

Related U.S. Application Data
(63) Continuation-in-part of application No. 18/184,971, filed on Mar. 16, 2023.

(30) Foreign Application Priority Data
Mar. 16, 2022 (IE) S2022/0048
May 8, 2023 (IE) S2023/0142
Aug. 23, 2023 (IE) S2023/0344

(51) Int. Cl. A61M 13/00; A61B 1/00

(52) U.S. Cl. CPC A61M 13/003; A61B 1/00002; A61M 2205/3344

(57) ABSTRACT

An insufflating system (201) comprises an insufflator (204) and an endoscope (205). The insufflator (204) comprises a flow control valve (217) for supplying insufflating gas to a cavity of a subject, and an isolating valve (222) for applying a vacuum to the cavity, both of which are connected to an instrument channel (208) of the endoscope (205) through a tube set (230). A pressure sensor (224) monitors cavity pressure through the instrument channel (208). The insufflator (201) is operable in a first operating mode in which a microprocessor (221) operates the flow control valve (217) to maintain the cavity pressure at a set pressure, and on the cavity pressure exceeding a predefined upper pressure, the microprocessor (221) operates the isolating valve (222) for applying vacuum to the cavity, and for further second time periods until the cavity pressure returns to the set pressure. The insufflator (204) is operable in a second operating mode in which the microprocessor (221) alternately and sequentially operates the flow control valve (217) and the isolating valve (222) to apply sequential pressure/vacuum cycles to the cavity in order to withdraw smoke generated in the cavity, while maintaining the cavity pressure within plus or minus 1 mmHg or 2 mmHg of the set pressure.

### FIELD OF THE INVENTION

The present invention relates to an insufflator for insufflating a cavity in the body of a human or animal subject, and the invention also relates to an insufflating system comprising an insufflator and an endoscope for insufflating a cavity in the body of a human or animal subject. Additionally, the invention relates to a method for insufflating a cavity in the body of a human or animal subject.

### BACKGROUND TO THE INVENTION

The term “cavity” is used in this specification, and the claims appended hereto to include any cavity, vessel, lumen or organ, and any cavity in any vessel, lumen or organ in the body of a human or animal subject which may be insufflated.

As surgical procedures have evolved, there has been an increasing move to perform procedures using an endoscope or a colonoscope. Hereinafter, through this specification and the claims appended hereto, the term “endoscope” is used to include an endoscope and a colonoscope. The key benefit of endoscopic procedures is that no incision has to be made on the skin of a patient, with the endoscope being introduced through a natural body orifice, such as the mouth or anus. Some basic endoscopic procedures, for example, removing small polyps in the colon, can be performed using endoscopic tools which pass through channels, generally, an instrument channel or channels within the endoscope. Other advanced procedures utilise overtubes through which the endoscope passes. These overtubes contain instrumentation and channels themselves, which are used in performing procedures, such as transoral incisionless fundoplication (TIF) procedures for treating gastroesophageal reflux from Endogastric Solutions. As these procedures evolve, so has the need to improve insufflating, namely, the introduction of gas to creating a working space for an endoscopist in the cavity in which the procedure is being carried out, and in particular, for the removal of smoke generated in the cavity in, for example, a low pressure dissecting procedure. In general, since only one channel is available in an endoscope for both delivery of insufflating gas to the cavity and the withdrawal of smoke and other gases from the cavity, the removal of smoke during a low pressure dissecting procedure or other such smoke generating procedures, is particularly challenging. Additionally, the removal of high pressure gases generated in the cavity or introduced into the cavity during an endoscope procedure, is also challenging.

Additionally, in traditional endoscope insufflating systems insufflating gas is delivered through a dedicated insufflating channel within the endoscope. The diameter of such insufflating channels is typically 1 mm or less, which in general, limits the maximum flow rate at which insufflating gas may be delivered through such endoscopes, to between 2 litres per minute and 3 litres per minute. However, when it is difficult to seal a surgical working space, leakage of insufflating gas can be high. For example, leakage of insufflating gas through the mouth or through the anus may be as high as 15 litres per minute to 20 litres per minute. Thus, it is challenging to achieve satisfactory insufflation at typical pressures in the range of 8 mmHg to 15 mmHg with such high leakage rates of insufflating gas.

Recent innovations involve the use of an endoscope-mounted tubeset END-200 from Palliare Ltd., which is the subject of Published PCT Patent Application Specification No. WO 2021/209983 which monitors pressure and adjusts flow to achieve a constant desired target insufflation pressure. This provides an endoscopic surgeon with the same quality of insufflation as that available for laparoscopic (or “key-hole”) surgery.

One problem that arises with endoscopic surgery systems which employ overtubes is that there is insufficient space between the endoscope and the overtube to locate a tube, such as the tubset END-200 of Palliare Ltd., between the endoscope and the overtube, and due to the relatively large outer diameter of overtubes, in general, it is not feasible to mount such a tube on the outside of such overtubes to support latest generation endoscopic insufflation technologies. A further requirement for such surgical procedures is the need to not only insufflate the working space, but to periodically desufflate the working space, to reduce the pressure to a lower pressure for example in the range 0 mmHg to 8 mmHg, to allow tissue to be more easily grabbed and sutured, which is easier to do if the surface being grabbed is not taut.

There is therefore a need for an insufflator for supplying insufflating gas from a source of insufflating gas to inflate a cavity in a human or animal subject, and there is also a need for an endoscope, and an insufflating system which addresses at least some of these problems. There is also a need for a method for insufflating a cavity in a human or animal subject, and there is a need for a method for carrying out a procedure in a cavity in the body of a human or animal subject which also addresses at least some of these problems.

There is therefore a need for an insufflator which addresses at least some of these problems, and there is also a need for an insufflating system which addresses at least some of these problems. Further, there is a need for a method for insufflating a cavity in the body of a human or animal subject which addresses at least some of these problems.

The present invention is directed towards providing such an insufflator, such an insufflating apparatus and such a method.

### SUMMARY OF THE INVENTION

According to the invention there is provided an insufflator for insufflating a cavity in the body of a human or animal subject, the insufflator comprising a first control means for controlling the supply of insufflating gas to the cavity, a second control means for controlling application of a vacuum to the cavity, a pressure monitoring means for monitoring pressure in the cavity (cavity pressure) and for producing a signal indicative of the cavity pressure, and a signal processor configured to read the signal from the pressure monitoring means and to control the first and second controls means in response to the signal read from the pressure monitoring means, wherein the signal processor is configured to selectively operate the insufflator in selectable ones of a first operating mode and a second operating mode, and in the first operating mode the signal processor is configured to operate the first control means in response to the signal read from the pressure monitoring means to control the supply of insufflating gas to the cavity for maintaining the cavity pressure substantially at a set pressure, and on the signal read from the pressure monitoring means being indicative of the cavity pressure exceeding a predefined upper pressure greater than the set pressure, the signal processor is configured to operate the second control means to apply a vacuum to the cavity until the cavity pressure falls to the set pressure, or to apply a vacuum to the cavity for a first predefined time period, and in the second operating mode, the signal processor is configured to control the first and second control means to sequentially and alternately supply insufflating gas to the cavity and to apply a vacuum to the cavity in a plurality of sequential pressure/vacuum cycles.

In one embodiment of the invention the first predefined time period lies in the range of 1 second to 5 seconds, and preferably, in the range of 2 seconds to 4 seconds, and ideally, the first predefined time period is approximately 3 seconds.

In another embodiment of the invention the signal processor is responsive to failure of the cavity pressure falling to or below the set pressure at the end of the first predefined time period to operate the second control means to apply the vacuum to the cavity for at least one second time period.

In another embodiment of the invention the signal processor is responsive to failure of the cavity pressure falling to or below the set pressure at the end of each second time period to operate the second control means to apply the vacuum to the cavity for another second time period.

In another embodiment of the invention the signal processor is programmed to compute the time duration of the first one of the second time periods as a function of the difference between the set pressure and the cavity pressure at the end of the first predefined time period.

In another embodiment of the invention the signal processor is programmed to compute the time duration of the first one of the second time periods as a function of the rate at which the cavity pressure dropped during the first predefined time period.

In another embodiment of the invention the signal processor is programmed to compute the time duration of the second and each subsequent one of the second time periods as a function of the difference between the set pressure and the cavity pressure at the end of the previous one of the second time periods.

In a further embodiment of the invention the signal processor is programmed to compute the time duration of the second one and each subsequent one of the second time periods as a function of the rate at which the cavity pressure dropped during the previous one of the second time periods.

In one embodiment of the invention the time duration of each second time period is shorter than the time duration of the first predefined time period, and preferably, the time duration of each subsequent second time period is shorter than the time duration of the immediately previous second time period.

In an alternative embodiment of the invention the time duration of each one of the second time periods is the same as the time duration of the first predefined time period.

In another embodiment of the invention the signal processor is programmed to operate the second control means to terminate the application of the vacuum to the cavity at the end of each one of the second time periods, and to read the signal from the pressure monitoring means.

In another embodiment of the invention the signal processor is programmed to operate the second control means to terminate the application of the vacuum to the cavity at the end of the first predefined time period and to read the signal from the pressure monitoring means.

In another embodiment of the invention in the first operating mode of the insufflator the signal processor is responsive to the cavity pressure exceeding the predefined upper pressure to operate the first control means to terminate the supply of insufflating gas to the cavity until the cavity pressure falls to or below the set pressure.

In one embodiment of the invention the predefined upper pressure lies in the range of 0.5 mmHg to 5 mmHg above the set pressure, and preferably, the predefined upper pressure lies in the range of 0.5 mmHg to 4 mmHg above the set pressure, and preferably, in the range of 1 mmHg to 4 mmHg above the set pressure, although, in some embodiments of the invention the predefined upper pressure may be approximately 1 mmHg above the set pressure.

In one embodiment of the invention in the second operating mode, the signal processor is programmed to operate the first and second control means during each pressure/vacuum cycle to maintain the cavity pressure within a predefined pressure range about the set pressure.

In one embodiment of the invention the predefined pressure range about the set pressure lies in the range of 3 mmHg above the set pressure and 3 mmHg below the set pressure. Preferably, the predefined pressure range about the set pressure lies in the range of 2 mmHg above the set pressure and 2 mmHg below the set pressure. Advantageously, the predefined pressure range above the set pressure lies in the range of 1 mmHg above the set pressure and 1 mmHg below the set pressure.

In another embodiment of the invention in the second operating mode, the signal processor is programmed to operate the first control means to supply the insufflating gas to the cavity for a predefined gas supply time period in each pressure/vacuum cycle.

In another embodiment of the invention in the second operating mode, the signal processor is programmed to operate the second control means to apply vacuum to the cavity for a predefined vacuum application time period in each pressure/vacuum cycle.

Preferably, each predefined vacuum application time period lies in the range of 1 second to 5 seconds, and advantageously, each predefined vacuum application time period lies in the range of 2 seconds to 4 seconds, and ideally, each predefined vacuum application time period is approximately 3 seconds.

In another embodiment of the invention each predefined gas supply time period lies in the range of 4 seconds to 10 seconds, and preferably, each predefined gas supply time period lies in the range of 6 seconds to 8 seconds, and ideally, each predefined gas supply time period is approximately 7 seconds.

In another embodiment of the invention the duration of the predefined vacuum application time period of each pressure/vacuum cycle lies in the range of one eighth to one half of the duration of each pressure/vacuum cycle, and preferably, the duration of the predefined vacuum application time period is approximately one third of the duration of each pressure/vacuum cycle.

In one embodiment of the invention in the second operating mode the signal processor is programmed to operate the second control means to prevent vacuum being applied to the cavity during the predefined gas supply time period of each pressure/vacuum cycle, and preferably, the signal processor is programmed to operate the first control means to prevent insufflating gas being supplied to the cavity during the predefined vacuum application time period of each pressure/vacuum cycle.

In one embodiment of the invention in the first operating mode the signal processor is programmed to operate the second control means to prevent vacuum being applied to the cavity when the first control means is being operated to supply insufflating gas to the cavity

In another embodiment of the invention in the first operating mode the signal processor is programmed to operate the first control means to prevent insufflating gas being supplied to the cavity when the second control means is being operated to apply a vacuum to the cavity.

Preferably, in the first operating mode the signal processor is responsive to the signal from the pressure monitoring means being indicative of the cavity pressure falling to or below the set pressure to operate the first control means to supply insufflating gas to the cavity for maintaining the cavity pressure at the set pressure.

In one embodiment of the invention the signal processor is programmed to be responsive to the signal read from the pressure sensing means being indicative of the cavity pressure having fallen to a predefined lower pressure lower than the set pressure during operation of the second control means applying vacuum to the cavity, to operate the second control means to terminate application of vacuum to the cavity, and to operate the first control means to supply insufflating gas to the cavity until the cavity pressure returns to the set pressure.

In another embodiment of the invention the predefined lower pressure lies in the range of 2 mmHg to 10 mmHg below the set pressure. Preferably, the predefined lower pressure lies in the range of 2 mmHg to 8 mmHg below the set pressure. Advantageously, the predefined lower pressure lies is approximately 6 mmHg below the set pressure.

In one embodiment of the invention the set pressure lies in the range of 2 mmHg to 15 mmHg, and preferably, the set pressure lies in the range of 5 mmHg to 15 mmHg, and ideally, the set pressure is approximately 10 mmHg.

In another embodiment of the invention the set pressure is selectable.

In another embodiment of the invention the insufflator comprises a first outlet port communicating with the first control means for accommodating insufflating gas therethrough to the cavity.

In another embodiment of the invention the insufflator comprises a second outlet port communicating with the second control means through which vacuum is applied to the cavity.

In another embodiment of the invention the insufflator comprises a third port for communicating the pressure monitoring means with the cavity. Alternatively, the pressure monitoring means is connected to the first outlet port or to the second outlet port. In a further embodiment of the invention the pressure monitoring means is selectively connectable to the third port or to one or both of the first or second outlet ports.

In one embodiment of the invention the insufflator comprises an insufflating gas source communicating with the first control means. Preferably, the insufflating gas source comprises a pressurised container containing the pressurised insufflating gas.

In an alternative embodiment of the invention the insufflator comprises a first inlet port communicating with the first control means for receiving insufflating gas from an external insufflating gas source thereof.

Preferably, the first control means comprises a flow control valve or a flow controller.

In one embodiment of the invention the second control means comprises a vacuum pump.

In an alternative embodiment of the invention the insufflator comprises a second inlet port communicating with the second control means for coupling the insufflator to an external vacuum source.

In one embodiment of the invention the second control means comprises an isolating valve selectively and alternately operable in an isolating state isolating the second outlet port from the second inlet port, and in a communicating state communicating the second outlet port with the second inlet port for applying vacuum from the external vacuum source to the second outlet port.

In another embodiment of the invention the pressure monitoring means comprises a pressure sensor, and preferably, the pressure monitoring means is located in the insufflator.

In another embodiment of the invention an interface means is provided communicating with the signal processor for selecting and entering the operating mode of the insufflator, and preferably, the interface means comprises at least one button switch for selecting each one of the first and second operating modes.

In another embodiment of the invention the signal processor is programmed to operate the insufflator in the first operating mode as a default operating mode.

In one embodiment of the invention a filter is provided for filtering gases drawn from the cavity during application of a vacuum to the cavity. Preferably, the filter comprises a bacterial-viral filter, and advantageously, the filter comprises a second activated carbon filter layer.

In one embodiment of the invention the filter is located between the cavity and the insufflator.

In another embodiment of the invention a water collecting means is provided for collecting water entrained in gases drawn from the cavity during application of a vacuum to the cavity, and preferably, the water collecting means is located between the filter and the cavity.

In one embodiment of the invention the water collecting means comprises a water trap.

The invention also provides an insufflating system for insufflating a cavity in the body of a human or animal subject, the insufflating system comprising an insufflator comprising a first control means for controlling the supply of insufflating gas to a cavity in the body of a subject for insufflating the cavity to a set pressure, and a second control means for applying a vacuum to the cavity, an endoscope comprising a channel capable of accommodating an insufflating gas to the cavity of the subject, and a connecting means connecting the insufflator to the said channel of the endoscope for alternately applying insufflating gas and a vacuum to the cavity through the said channel of the endoscope.

In one embodiment of the invention the first control means and the second control means are configured for sequentially supplying insufflating gas and applying the vacuum to the said channel of the endoscope.

In another embodiment of the invention the first control means and the second control means are configured to supply insufflating gas and to apply the vacuum in sequential insufflating gas/vacuum cycles to the said channel of the endoscope.

Preferably, the first control means is configured for supplying insufflating gas to the said channel of the endoscope during each insufflating gas/vacuum cycle for a predefined gas supply time period, and the second control means is configured for applying the vacuum to the said channel of the endoscope during each insufflating gas/vacuum cycle for a predefined vacuum application time period.

In one embodiment of the invention the second control means is responsive to the cavity pressure exceeding the set pressure or a predefined upper pressure greater than the set pressure for applying the vacuum to the said channel of the endoscope.

In one embodiment of the invention the second control means is responsive to the cavity pressure exceeding the preset pressure or the predefined upper pressure for applying the vacuum to the said channel of the endoscope for one predefined vacuum application time period.

Preferably, the second control means is configured to apply the vacuum to the said channel of the endoscope until the cavity pressure is reduced to or below the set pressure or to a predefined lower pressure below the set pressure.

In one embodiment of the invention the connecting means comprises a first port being configured for communicating with the said channel of the endoscope, a second port being configured for communicating with the first control means, and a third port being configured for communicating with the second control means.

In another embodiment of the invention the connecting means comprises a Y-connector.

Preferably, the insufflator comprises a first outlet port and a second outlet port, and the first control means communicates with the first outlet port, and the second control means communicates with the second outlet port.

Advantageously, the second port of the connecting means is configured for communicating with the first outlet port, and the third port of the connecting means is configured for communicating with the second outlet port.

In one embodiment of the invention the insufflator comprises a pressure monitoring means adapted for selectively connecting to the said channel of the endoscope or to another channel of the endoscope for monitoring the pressure in the cavity.

In another embodiment of the invention the channel of the endoscope capable of accommodating insufflating gas to the cavity of a subject comprises one of an instrument channel of the endoscope, an insufflating channel of the endoscope, a vacuum channel of the endoscope, or a channel defined by a tubular member attached and extending to the endoscope externally thereof.

In one embodiment of the invention the vacuum applied to the cavity by the second control means is adapted for drawing smoke and/or gases from the cavity.

Preferably, the first control means is configured to apply insufflating gas to the cavity for maintaining the cavity pressure at the set pressure.

Further the invention provides an insufflating system for insufflating a cavity in the body of a human or animal subject, the insufflating system comprising the insufflator according to the invention, and an endoscope comprising a channel capable of accommodating an insufflating gas to the cavity of the subject, and a connecting means connecting the insufflator to the said channel for alternately applying the insufflating gas and the vacuum to the said channel of the endoscope.

In one embodiment of the invention the connecting means is adapted for connecting to the first control means and to the second control means and to the said channel of the endoscope.

In another embodiment of the invention the connecting means comprises a first port being configured for communicating with the said channel of the endoscope, a second port being configured for communicating with the first control means, and a third port configured for communicating with the second control means.

Preferably, the second port of the connecting means is adapted for coupling to the first outlet port of the insufflator, and the third port of the connecting means is adapted for coupling to the second outlet port of the insufflator.

In one embodiment of the invention the connecting means comprises a Y-connector.

The invention also provides an insufflator for insufflating a cavity in the body of a human or animal subject, the insufflator comprising a first control means for controlling the supply of insufflating gas to the cavity, a second control means for controlling application of a vacuum to the cavity, and a signal processor programmed to sequentially and alternately operate the first control means to supply insufflating gas to the cavity, and the second control means to apply a vacuum to the cavity in a plurality of sequential pressure/vacuum cycles for maintaining an intermittent flow of insufflating gas through the cavity to withdraw smoke and/or other undesirable gases from the cavity.

Preferably, the signal processor is programmed to operate the first control means to supply insufflating gas to the cavity for a predefined gas supply time period in each pressure/vacuum cycle.

Advantageously, the signal processor operates the second control means to apply vacuum to the cavity for a predefined vacuum application time period in each pressure/vacuum cycle.

Additionally, the invention provides a method for insufflating a cavity in the body of a human or animal subject with an insufflator operable in selectable first and second operating modes, the method comprising monitoring the pressure in the cavity (cavity pressure), operating the insufflator in the first operating mode for insufflating the cavity to maintain the cavity pressure at a set pressure, and in response to the cavity pressure exceeding the set pressure or a predefined upper pressure greater than the set pressure operating the insufflator to apply a vacuum to the cavity until the cavity pressure falls to the set pressure, or operating the insufflator to apply a vacuum to the cavity for a first predefined time period, and operating the insufflator in the second operating mode for sequentially and alternately supplying insufflating gas to the cavity and applying a vacuum to the cavity in a plurality of sequential pressure/vacuum cycles.

In one embodiment of the invention on failure of the cavity pressure to fall to or below the set pressure at the end of the first predefined time period, the insufflator is operated to apply the vacuum to the cavity for at least one second time period.

In another embodiment of the invention on failure of the cavity pressure to fall to or below the set pressure at the end of each second time period the insufflator is operated to apply the vacuum to the cavity for another second time period.

In another embodiment of the invention the time duration of the first one of the second time periods is computed as a function of the difference between the set pressure and the cavity pressure at the end of the first predefined time period.

In another embodiment of the invention the time duration of the first one of the second time periods is computed as a function of the rate at which the cavity pressure dropped during the first predefined time period.

In another embodiment of the invention the time duration of the second and each subsequent one of the second time periods is computed as a function of the difference between the set pressure and the cavity pressure at the end of the previous one of the second time periods.

In one embodiment of the invention the time duration of the second and each subsequent one of the second time periods is computed as a function of the rate at which the cavity pressure dropped during the previous one of the second time periods.

In one embodiment of the invention the application of the vacuum to the cavity is terminated at the end of each one of the second time periods, and the cavity pressure is monitored.

In another embodiment of the invention the application of the vacuum to the cavity is terminated at the end of the first predefined time period and the cavity pressure is monitored.

In another embodiment of the invention in the first operating mode on the cavity pressure exceeding the set pressure or the predefined upper pressure, the supply of insufflating gas to the cavity is terminated until the cavity pressure falls to or below the set pressure.

In one embodiment of the invention the second operating mode, the cavity pressure is maintained within a predefined pressure range about the set pressure during each pressure/vacuum cycle.

In one embodiment of the invention insufflating gas is supplied to the cavity during each pressure/vacuum cycle for a predefined gas supply time period.

In another embodiment of the invention vacuum is applied to the cavity during each pressure/vacuum cycle for a predefined vacuum application time period.

Preferably, the supply of insufflating gas to the cavity is interrupted during the predefined vacuum application time period of each pressure/vacuum cycle.

In another embodiment of the invention the application of vacuum to the cavity is interrupted during the predefined gas supply time period of each pressure/vacuum cycle.

In another embodiment of the invention the first operating mode the application of vacuum to the cavity is prevented when the first insufflating gas is being supplied to the cavity.

In another embodiment of the invention in the first operating mode the supply of insufflating gas to the cavity is prevented when vacuum is being supplied to the cavity.

In another embodiment of the invention in the first operating mode on the cavity pressure falling to or below the set pressure insufflating gas is supplied to the cavity for maintaining the cavity pressure at the set pressure.

In another embodiment of the invention application of vacuum to the cavity is terminated on the cavity pressure falling to our below a predefined lower pressure lower than the set pressure, and insufflating gas is supplied to the cavity until the cavity pressure returns to the set pressure.

In one embodiment of the invention insufflating gas is supplied to the cavity and the vacuum is applied to the cavity from the insufflator through a channel in an endoscope capable of accommodating insufflating gas to the cavity.

In one embodiment of the invention the said channel of the endoscope is connected to a first control means of the insufflator which controls the supply of insufflating gas to the cavity for accommodating insufflating gas to the cavity.

In another embodiment of the invention the said channel of the endoscope is connected to a second control means of the insufflator which controls the application of the vacuum to the cavity for applying the vacuum to the cavity.

In another embodiment of the invention the said channel of the endoscope is connected to the first and second control means through a connecting means.

Preferably, the cavity pressure is monitored through the said channel of the endoscope. Alternatively, the cavity pressure is monitored through a channel of the endoscope other than the said channel.

In one embodiment of the invention the endoscope is inserted into the cavity through a bodily orifice of the subject.

Preferably, gases drawn from the cavity during application of vacuum to the cavity are filtered. Advantageously, the gases drawn from the cavity by the application of vacuum to the cavity are filtered through a bacterial-viral filter. Preferably, the filter is connected between the said channel of the endoscope and the insufflator.

In another embodiment of the invention water entrained in gases drawn from the cavity by the application of vacuum to the cavity is collected in a water collecting means. Preferably, the water collecting means is located between the said channel of the endoscope and the insufflator.

Further the invention provides a method for insufflating a cavity in the body of a human or animal subject comprising sequentially and alternately supplying insufflating gas to the cavity and applying a vacuum to the cavity in a plurality of sequential pressure/vacuum cycles for maintaining an intermittent flow of insufflating gas through the cavity to withdraw smoke and/or other undesirable gases from the cavity.

The invention further provides a method for insufflating a cavity in the body of a human or animal subject and for controlling the pressure at which the cavity is insufflated substantially at a set pressure, the method comprising inserting an endoscope into the cavity, connecting a channel of the endoscope capable of accommodating an insufflating gas to the cavity to an insufflator, and sequentially and alternately applying insufflating gas to the said channel of the endoscope for delivery to the cavity and applying a vacuum to the said channel of the endoscope in sequential pressure/vacuum cycles for withdrawing the smoke or the gas from the cavity with the cavity maintained substantially at the set pressure.

Further, the invention provides an insufflator for supplying insufflating gas from a source of insufflating gas to insufflate a cavity in a human or an animal subject, the insufflator comprising a pressure regulating means adapted to control the pressure at which the insufflating gas is supplied from the insufflator to the cavity at respective first and second pressures, the second pressure being lower than the first pressure, a first receiving means adapted to receive a remotely generated signal indicative of a change pressure request to change the pressure at which the insufflating gas is being supplied to the cavity from the current one of the first and second pressures to the other one of the first and second pressures, and to produce a change signal in response to reception of the signal indicative of a change pressure request, and a control means responsive to the change signal produced by the first receiving means to operate the pressure regulating means to alter the pressure at which the insufflating gas is being supplied to the cavity from the current one of the first and second pressures at which the insufflating gas is being supplied to the other one of the first and second pressures thereof.

In one embodiment of the invention the first receiving means is adapted to receive the signal indicative of the change pressure request as any one or more of wirelessly, acoustically, and by wire.

In another embodiment of the invention the first receiving means comprises any one or more of a wireless receiver, a microphone, a hardwire connector configured for connecting to a wire carrying the signal indicative of the change pressure request, and a pneumatic sensor configured for receiving a pneumatic signal indicative of the change pressure request.

In another embodiment of the invention the first receiving means comprises a voice recognition module adapted to interpret a human voice sound indicative of a change pressure request.

In one embodiment of the invention the first receiving means is adapted to receive the signal indicative of a change pressure request from one or more of a foot operated electrical switch, a hand operated electrical switch, a foot operated pneumatic switch, and a hand operated pneumatic switch.

In another embodiment of the invention the first receiving means is adapted to receive the signal indicative of the change pressure request as any one or more of a signal indicative of entry of an instrument into the cavity in the subject, a signal indicative of the commencement of operation of an instrument in the cavity in the subject, a signal indicative of the operation of the instrument in the cavity in the subject, and a signal indicative of termination of operation of an instrument in the cavity.

In another embodiment of the invention the first receiving means is adapted to receive the signal indicative of the commencement of operation of an instrument in the cavity, the signal indicative of the operation of an instrument in the cavity, and the signal indicative of termination of operation of an instrument in the cavity from one or more of a sensor on the instrument adapted to monitor operation thereof, or from an instrument controller adapted to control the operation of the instrument.

Preferably, the pressure regulating means is responsive to a signal indicative of the pressure in the cavity in the subject for maintaining the pressure of the insufflating gas in the cavity at the current one of the first and second pressures at which the insufflating gas is to be supplied to the cavity.

Advantageously, the pressure regulating means is adapted to control the rate of flow of the insufflating gas to the cavity in the subject to maintain the pressure in the cavity at the current one of the first and second pressures at which the insufflating gas is to be supplied to the cavity.

In one embodiment of the invention the pressure regulating means is adapted to alter the rate of flow of the insufflating gas to the cavity in the subject for altering the pressure in the cavity from the current one of the first and second pressures at which the insufflating gas is being supplied to the cavity to the other one of the first and second pressures.

Preferably, the pressure regulating means is adapted to increase the rate of flow of the insufflating gas to the cavity in the subject to increase the pressure at which the insufflating gas is being supplied to the cavity from the second pressure to the first pressure.

Preferably, the pressure regulating means is adapted to decrease the rate of flow of the insufflating gas to the cavity in the subject to decrease the pressure at which the insufflating gas is being supplied to the cavity from the first pressure to the second pressure.

Advantageously, the pressure regulating means is adapted to reduce the rate of flow of insufflating gas to the cavity in the subject to approximately zero flow rate to decrease the pressure at which the insufflating gas is being supplied to the cavity from the first pressure to the second pressure.

In one embodiment of the invention the pressure regulating means comprises a flow controller for controlling the rate of flow of the insufflating gas being supplied from the insufflator.

Preferably, the flow controller is operable under the control of the control means for controlling the rate of flow of the insufflating gas from the insufflator.

In one embodiment of the invention the insufflator further comprises a pressure sensor adapted to monitor pressure in the cavity in the subject and for producing a signal indicative of the pressure in the cavity.

In another embodiment of the invention the control means is responsive to the signal produced by the pressure sensor indicative of the pressure in the cavity for operating the pressure regulating means to control the flow rate of the insufflating gas from the insufflator for maintaining the pressure in the cavity at the current one of the first and second pressures at which the insufflating gas is to be supplied to the cavity.

In another embodiment of the invention the insufflator further comprises a vacuum system or is adapted for coupling to an external vacuum for withdrawing insufflating gas from the cavity in the subject, the vacuum system being connected to the cavity under the control of the control means in response to the change signal when the current pressure at which the cavity is being insufflated is the first pressure and the pressure in the cavity is to be reduced to the second pressure.

Preferably, the vacuum system is operated under the control of the control means in response to the signal indicative of the pressure in the cavity read from the pressure sensor until the pressure in the cavity has been reduced to the second pressure.

In one embodiment of the invention vacuum applied to the cavity is terminated on the pressure in the cavity falling to the second pressure.

In another embodiment of the invention a vacuum control valve means is provided in the insufflator, the vacuum control valve means being operable under the control of the control means in response to the signal indicative of a change pressure request when the pressure regulating circuit is supplying the insufflating gas at the first pressure for applying a vacuum from the vacuum system to the cavity.

Preferably, the control means is responsive to the signal read from the pressure sensor indicative of the pressure in the cavity being indicative of the pressure in the cavity falling to the second pressure for operating the vacuum control valve means to disconnect the vacuum system from the cavity.

Advantageously, the vacuum control valve means and the pressure regulating means are operable under the control of the control means so that when the vacuum control valve means is operating to apply a vacuum to the cavity, the supply of insufflating gas to the cavity is interrupted.

In one embodiment of the invention the vacuum system comprises a vacuum pump.

In one embodiment of the invention the pressure value of at least one of the first and second pressures is selectable.

Preferably, the first pressure value is selectable within a range of pressure values between 8 mmHg to 15 mmHg. Advantageously, the second pressure value is selectable within a range of pressure values between 0 mmHg to 8 mmHg.

In one embodiment of the invention the insufflator further comprises a first output port and a second output port, and an output valve means through which insufflating gas is delivered from the pressure regulating circuit to the first and second output ports, the output valve means being operable in at least two states, a first state with the first output port communicating with the pressure regulating circuit for delivering insufflating gas through the first output port and the second output port isolated from the pressure regulating circuit, and a second state with the second output port communicating with the pressure regulating circuit for delivering insufflating gas through the second output port and the first output port isolated from the pressure regulating circuit.

In another embodiment of the invention the output valve comprises one of a solenoid valve, a motor controlled valve, and a pneumatically controlled valve.

Preferably, the control means is responsive to a remotely generated signal indicative of an instrument in or being entered into an instrument channel of an endoscope for switching the operation of the output valve means from the first state to the second state.

In one embodiment of the invention the control means is responsive to a signal indicative of an instrument being withdrawn from an instrument channel of an endoscope for switching operation of the output valve means from the second state to the first state.

In another embodiment of the invention the output valve means is operable in a third state with the first and second output ports communicating with the pressure regulating circuit for simultaneously supplying insufflating gas through the first and second output ports.

Preferably, the output valve means is selectively operable under the control of the control means in the first state, the second state and the third state.

In one embodiment of the invention the output valve means is operable in a fourth state to isolate the first and second output ports simultaneously from the pressure regulating circuit to prevent delivery of insufflating gas therethrough.

Preferably, the output valve means is selectively operable under the control of the control means in the first state, the second state, the third state and the fourth state.

In one embodiment of the invention the control means is responsive to a signal indicative of back pressure of the insufflating gas at one of the first and second output ports through which insufflating gas is being supplied to the cavity or at the proximal end of an insufflating channel of an endoscope through which the insufflating gas is being supplied to the cavity in the subject for operating the output valve means from the one of the first and second states in which the output valve means is operating to the other one of the first and second states thereof.

In one embodiment of the invention the insufflator further comprises a first input port and a second input port, and an input valve means through which the first and second input ports communicate with the pressure sensor, the input valve means being operable in two states, a first state with the first input port communicating with the pressure sensor, and the second input port isolated from the pressure sensor, and a second state with the second input port communicating with the pressure sensor and the first input port isolated from the pressure sensor.

In one embodiment of the invention the input valve means is operable under the control of the control means in the first and second states.

Preferably, the control means is responsive to a signal indicative of an instrument in or being entered into an instrument channel of an endoscope for switching the operation of the input valve means from the first state to the second state.

Advantageously, the control means is responsive to a signal indicative of an instrument being withdrawn from an instrument channel of an endoscope for switching operation of the input valve means from the second state to the first state.

In another embodiment of the invention the insufflator comprises a second receiving means configured to receive the signal indicative of an instrument in or being entered into an instrument channel of an endoscope and the signal indicative of an instrument being withdrawn from an endoscope produced by an instrument sensor, whereby the signal indicative of an instrument in or being entered into an instrument channel of the endoscope and the signal indicative of an instrument being withdrawn from an instrument channel of an endoscope are produced wirelessly, by wire or acoustically, for example, by voice.

In one embodiment of the invention the second receiving means is configured to receive the signal indicative of an instrument in or being entered into an instrument channel of an endoscope wirelessly in a Bluetooth protocol.

The invention also provides an endoscope comprising an insufflating channel and an instrument channel, the instrument channel being adapted to accommodate an instrument therethrough and to accommodate insufflating gas therethrough for insufflating a cavity in the body of a human or animal subject.

In one embodiment of the invention the instrument channel is adapted to selectively and alternately accommodate an instrument therethrough and the insufflating gas.

In another embodiment of the invention the instrument channel is configured for selectively connecting to a supply of water, and preferably, the supply of water is provided for cleaning a lens adjacent a distal end of the endoscope of an imaging system of the endoscope.

In one embodiment of the invention the supply of water comprises a pressurised supply of water, and preferably, the instrument channel is configured for selectively connecting to the water supply when the instrument channel is adapted to accommodate the insufflating gas therethrough.

In one embodiment of the invention the proximal end of the insufflating channel of the endoscope is adapted to communicate with a pressure sensor, and preferably, a pressure sensor in an insufflator.

In another embodiment of the invention the insufflating channel is adapted to sealably communicate with the pressure sensor, and preferably, to communicate the pressure sensor with a cavity being insufflated through the endoscope.

In another embodiment of the invention the insufflating channel of the endoscope is configured to alternately communicate with an insufflator to receive and to accommodate the insufflating gas therethrough to a cavity to be insufflated, and communicate with the pressure sensor. Preferably, the insufflating channel of the endoscope is adapted to sealably communicate with the pressure sensor, and advantageously, the insufflating channel is adapted to communicate the pressure sensor with the cavity being insufflated.

In one embodiment of the invention a valve means is provided for alternately or simultaneously communicating the insufflating channel and the instrument channel of the endoscope with the pressure sensor.

In another embodiment of the invention the insufflating channel is adapted for selectively connecting to a supply of water, and preferably, a supply of water for cleaning a lens adjacent the distal end of the endoscope of an imaging system thereof.

Preferably, the supply of water comprises a pressurised supply of water, and advantageously, the insufflating channel is adapted for selectively connecting to the water supply when the insufflating channel is configured for accommodating insufflating gas therethrough.

In one embodiment of the invention the endoscope further comprises a directing means for directing water supplied through the instrument channel to the lens of the imaging system.

In one embodiment of the invention the directing means is urgeable through the instrument channel from the proximal end to the distal end thereof for directing water exiting from the distal end of the instrument channel to the lens of the imaging system.

In another embodiment of the invention the directing means comprises a deflecting element, and preferably, the deflecting element comprises any one of a spherical element, a conical element and a dish shaped element.

In another embodiment of the invention the deflecting means comprises the conical element, an apex of a cone face thereof facing in a proximal direction relative to the proximal and distal ends of the endoscope.

In another embodiment of the invention the deflecting element comprises the dish shaped element, the dish shaped element being concave when viewed through the instrument channel.

In another embodiment of the invention the directing means is mounted in or on the distal end of the endoscope.

In one embodiment of the invention the directing means is operable between a rest state and a deflecting state for directing water exiting from the instrument channel adjacent the distal end thereof to the lens of the imaging system.

In one embodiment of the invention the directing means is operable between the rest state and the deflecting state when the directing means is extending distally outwardly from the instrument channel.

In another embodiment of the invention the directing means comprises a deflecting element operable between a rest state and a deflecting state when the deflecting element has been urged distally outwardly from the distal end thereof.

In one embodiment of the invention the directing means is urgeable from the rest state to the deflecting state on exiting the instrument channel adjacent the distal end thereof, and advantageously, the directing means is resiliently biased into the deflecting state, and preferably, is spring urged into the deflecting state.

In another embodiment of the invention the directing means is operable between the rest state and the deflecting state by an operating means to which the deflecting means is connected.

In one embodiment of the invention an instrument sensor is provided for detecting an instrument in or being entered into the instrument channel of the endoscope and for producing a signal indicative of an instrument in or being entered into the instrument channel.

In one embodiment of the invention an instrument sensor is provided for detecting an instrument in or being entered into the instrument channel of the endoscope and for producing a signal indicative of an instrument in or being entered into the instrument channel. Preferably, the instrument sensor is located adjacent a proximal end of the instrument channel, and advantageously, the instrument sensor is located in or adjacent the endoscope.

In another embodiment of the invention the instrument sensor comprises a transmitter for transmitting the signal indicative of an instrument in or being entered into the instrument channel, and the transmitted signal is adapted for reception by an insufflator to switch delivery of the insufflating gas from the instrument channel to an insufflating channel of the endoscope.

In another embodiment of the invention the instrument sensor is configured to produce a signal indicative of an instrument being withdrawn from the instrument channel of the endoscope and to transmit the signal indicative of an instrument being withdrawn from the instrument channel for reception by an insufflator.

Preferably, the signal indicative of an instrument being withdrawn from the instrument channel is defined by the termination of the transmission of the signal indicative of an instrument in or being entered into the instrument channel by the instrument sensor.

In one embodiment of the invention the instrument sensor comprises a transmitter for transmitting the signals produced by the instrument sensor, and in another embodiment of the invention the transmitted signal is adapted for reception by an insufflator.

Preferably, the instrument sensor comprises a wireless transmitter, and preferably, the wireless transmitter is adapted for transmitting a wireless signal in a Bluetooth protocol.

Additionally, the invention provides an insufflating system comprising an insufflator and an endoscope, the endoscope being connected to the insufflator with an instrument channel of the endoscope being configured to receive insufflating gas from the insufflator for delivery therethrough to insufflate a cavity in a human or animal subject.

In one embodiment of the invention the insufflator comprises an insufflator according to the invention.

In another embodiment of the invention the endoscope comprises an endoscope according to the invention.

In one embodiment of the invention the insufflator comprises a pressure regulating means adapted to control the pressure at which the insufflating gas is supplied from the insufflator to the cavity through the endoscope at respective first and second pressures, the second pressure being lower than the first pressure, a first receiving means adapted to receive a remotely generated signal indicative of a change pressure request to change the pressure at which the insufflating gas is being supplied from the current one of the first and second pressures to the other one of the first and second pressures, and to produce a change signal in response to reception of the signal indicative of a change pressure request, and a control means responsive to the change signal produced by the first receiving means for operating the pressure regulating means to alter the pressure at which the insufflating gas is being supplied to the cavity from the current one of the first and second pressure to the other one of the first and second pressures thereof.

In one embodiment of the invention the instrument channel of the endoscope is selectively connectable to the insufflator to receive insufflating gas from the insufflator at the one of the first and second pressures.

In another embodiment of the invention an insufflating channel of the endoscope is selectively connectable to the insufflator to receive the insufflating gas from the insufflator at the one of the first and second pressures.

In one embodiment of the invention the instrument channel and the insufflating channel of the endoscope are selectively and alternately connectable to the insufflator to receive the insufflating gas from the insufflator at the one of the first and second pressures.

In one embodiment of the invention the instrument channel of the endoscope is connected to one of the first and second output ports of the insufflator.

In one embodiment of the invention the insufflator comprises a pressure sensor for monitoring the pressure in the cavity in the subject.

Preferably, the insufflating channel and the instrument channel of the endoscope are selectively connectable to the pressure sensor of the insufflator.

Preferably, the insufflating channel and the instrument channel of the endoscope are selectively and alternately connectable to the pressure sensor of the insufflator.

In one embodiment of the invention the insufflating channel of the endoscope is connected to the other one of the first and second output ports of the insufflator to that to which the instrument channel is connected.

In one embodiment of the invention the insufflating channel of the endoscope is connected to the pressure sensor of the insufflator through one of the first and second input ports of the insufflator.

Preferably, the instrument channel of the endoscope is connected to the other one of the first and second input ports of the insufflator to that to which the insufflating channel is connected.

In one embodiment of the invention the insufflator further comprises a pressure sensor adapted to monitor pressure in the cavity in the subject and for producing a signal indicative of the pressure in the cavity.

Preferably, the control means is responsive to the signal produced by the pressure sensor indicative of the pressure in the cavity for operating the pressure regulating means to control the flow rate of the insufflating gas from the insufflator for maintaining the pressure in the cavity at the current one of the first and second pressures at which the insufflating gas is to be supplied to the cavity.

In one embodiment of the invention the insufflator further comprises a vacuum system or is adapted for coupling to an external vacuum for withdrawing insufflating gas from the cavity in the subject, the vacuum system being adapted for connecting to a vacuum channel or the instrument channel of the endoscope under the control of the control means in response to the change signal when the current pressure at which the cavity is being insufflated is the first pressure and the pressure in the cavity is to be reduced to the second pressure.

In another embodiment of the invention the vacuum system is operated under the control of the control means in response to the signal indicative of the pressure in the cavity read from the pressure sensor until the pressure in the cavity in the subject has been reduced to the second pressure.

Preferably, vacuum applied to the cavity is terminated on the pressure in the cavity falling to the second pressure.

In another embodiment of the invention a vacuum control valve means is provided in the insufflator, the vacuum control valve means being operable under the control of the control means in response to the signal indicative of a change pressure request when the pressure regulating circuit is supplying the insufflating gas at the first pressure for applying a vacuum from the vacuum system to the cavity.

Preferably, the control means is responsive to the signal read from the pressure sensor indicative of the pressure in the cavity being indicative of the pressure in the cavity falling to the second pressure for operating the vacuum control valve means to disconnect the vacuum system from the cavity.

Advantageously, the vacuum control valve means and the pressure regulating means are operable under the control of the control means so that when the vacuum control valve means is operating to apply a vacuum to the cavity, the supply of insufflating gas to the cavity is interrupted.

Preferably, the vacuum system comprises a vacuum pump.

In another embodiment of the invention an instrument sensor is provided for detecting an instrument in or being entered into the instrument channel of the endoscope, and for producing a signal indicative of an instrument in or being entered into the instrument channel.

In another embodiment of the invention the control means of the insufflator is responsive to the signal indicative of the instrument in or being entered into the instrument channel for switching the insufflating gas from the instrument channel to the insufflating channel of the endoscope.

In another embodiment of the invention the control means of the insufflator is responsive to the signal indicative of an instrument in or being entered into the instrument channel for switching the connection of the pressure sensor from the insufflating channel to the instrument channel of the endoscope.

In another embodiment of the invention the insufflator comprises a vacuum system, and the vacuum system is connected to the vacuum channel of the endoscope or to the instrument channel of the endoscope.

In one embodiment of the invention the instrument channel is connected to the vacuum system of the insufflator through a vacuum control valve.

In another embodiment of the invention the instrument channel is selectively and alternately connected to the vacuum system and the pressure regulating means of the insufflator.

In another embodiment of the invention two pressure sensors are provided, one pressure sensor being configured for monitoring the pressure of the insufflating gas in the insufflating channel, and the other pressure sensor being configured for monitoring the pressure of the insufflating gas in the instrument channel of the endoscope. In one embodiment of the invention one or both of the pressure sensors are located in the insufflator, and in an alternative embodiment of the invention one or both of the pressure sensors are located in the endoscope.

In another embodiment of the invention one of the pressure sensors is located in the insufflator, and the other one of the pressure sensors is located in the endoscope. In embodiments of the invention where the two pressure sensors are provided, the first input valve may be dispensed with.

In another embodiment of the invention the insufflator further comprises a first output port configured for coupling to the instrument channel of the endoscope, and a second output port configured for coupling to the insufflating channel of the endoscope, and an output valve means through which insufflating gas is delivered from the pressure regulating circuit to the first and second output ports, the output valve means being operable in at least two states, a first state with the first output port communicating with the pressure regulating circuit for delivering insufflating gas through the first output port and the second output port isolated from the pressure regulating circuit, and a second state with the second output port communicating with the pressure regulating circuit for delivering insufflating gas through the second output port and the first output port isolated from the pressure regulating circuit.

Preferably, the output valve means is selectively and alternately operable under the control of the control means in the first and second states.

In another embodiment of the invention the control means is responsive to the signal indicative of an instrument in or being entered into the instrument channel of the endoscope for switching the operation of the output valve means from the first state to the second state.

Preferably, the control means is responsive to the signal indicative of an instrument being withdrawn from the instrument channel of the endoscope for switching operation of the output valve means from the second state to the first state.

Advantageously, the output valve means is operable in a third state with the first and second output ports communicating with the pressure regulating circuit for simultaneously supplying insufflating gas through the first and second output ports.

Preferably, the output valve means is selectively operable under the control of the control means in the first state, the second state and the third state.

Advantageously, the output valve means is operable in a fourth state to isolate the first and second output ports simultaneously from the pressure regulating circuit to prevent delivery of insufflating gas therethrough.

Advantageously, the output valve means is selectively operable under the control of the control means in the first state, the second state, the third state and the fourth state.

In one embodiment of the invention the control means is responsive to a signal indicative of back pressure of the insufflating gas at one of the first and second output ports through which insufflating gas is being supplied to the cavity or at the proximal end of an insufflating channel of an endoscope through which the insufflating gas is being supplied to the cavity in the subject for operating the output valve means from the one of the first and second states in which the output valve means is operating to the other one of the first and second states thereof.

In another embodiment of the invention the insufflator further comprises a first input port configured for coupling to the insufflating channel of the endoscope, and a second input port configured for coupling to the instrument channel of the endoscope, and an input valve means through which the first and second input ports communicate with the pressure sensor, the input valve means being operable in two states, a first state with the first input port communicating with the pressure sensor, and the second input port isolated from the pressure sensor, and a second state with the second input port communicating with the pressure sensor and the first input port isolated from the pressure sensor.

In one embodiment of the invention the input valve means is operable under the control of the control means in the first and second states.

In another embodiment of the invention the control means is responsive to the signal indicative of an instrument in or being entered into the instrument channel of the endoscope for switching the operation of the input valve means from the first state to the second state.

Preferably, the control means is responsive to the signal indicative of an instrument being withdrawn from the instrument channel of the endoscope for switching operation of the input valve means from the second state to the first state.

Further the invention provides a method for insufflating a cavity in a human or animal subject, the method comprising:
inserting an endoscope through a natural orifice in the human or animal subject into the cavity,
delivering insufflating gas into the cavity through an instrument channel of the endoscope, and
monitoring pressure in the cavity through an insufflating channel of the endoscope.

In one embodiment of the invention delivery of the insufflating gas through the endoscope is switched from the instrument channel to the insufflating channel in response to an instrument being entered into the instrument channel.

In another embodiment of the invention the monitoring of the pressure in the cavity is switched from being monitored through the insufflating channel to the instrument channel of the endoscope in response to an instrument being entered into the instrument channel.

In another embodiment of the invention the pressure in the cavity is monitored through the insufflating channel when the insufflating gas is being delivered to the cavity through the insufflating channel during a plurality of time spaced apart monitoring time periods during which delivery of insufflating gas to the cavity through the insufflating channel is paused.

Preferably, delivery of the insufflating gas through the insufflating channel is momentarily paused during each monitoring time period for monitoring the cavity pressure through the insufflating channel, and preferably, is paused during each monitoring time period for a period of 1 second to 2 seconds.

Preferably, the pressure in the cavity is monitored through the instrument channel when the insufflating gas is being delivered to the cavity through the instrument channel during a plurality of time spaced apart monitoring time periods during which delivery of insufflating gas to the cavity through the instrument channel is paused.

Advantageously, delivery of the insufflating gas through the instrument channel is paused for 1 second or 2 seconds during each predefined monitoring time period for monitoring the cavity pressure through the instrument channel.

In another embodiment of the invention water for cleaning a lens of an imaging system of the endoscope is delivered through the instrument channel when the insufflating gas is being delivered to the cavity through the insufflating channel of the endoscope.

Preferably, the water exiting the instrument channel adjacent the distal end thereof is directed towards the lens of the imaging system.

In another embodiment of the invention on an instrument being entered into the instrument channel, the pressure in the cavity is monitored through the instrument channel. Alternately, the pressure in the cavity may be monitored through the insufflating channel when the insufflating gas is being delivered to the cavity through the insufflating channel, and preferably, the pressure in the cavity is monitored while the delivery of insufflating gas to the cavity through the insufflating channel is paused.

In one embodiment of the invention the delivery of the insufflating gas through the insufflating channel is momentarily paused for monitoring the cavity pressure through the insufflating channel.

In another embodiment of the invention when the insufflating gas is being delivered through the instrument channel, pressurised water for cleaning a lens of an imaging system of the endoscope is delivered through the instrument channel, and advantageously, a directing means is provided for directing the pressurised water from the distal end of the instrument channel onto the lens.

Additionally, the invention provides a method for carrying out a procedure in a cavity in the body of a human or animal subject comprising:
insufflating the cavity at a first pressure or a second pressure, the second pressure being different to the first pressure,
commencing carrying out of the procedure with the cavity insufflated at the one of the first and second pressures,
during carrying out of the procedure altering the pressure at which the cavity is being insufflated from the one of the first and second pressures at which the cavity is currently being insufflated to the other one of the first and second pressures.

In one embodiment of the invention the pressure at which the cavity is being insufflated is altered at least twice during the carrying out of the procedure, and each time the pressure is altered, the pressure is altered from the one of the first and second pressures at which the cavity is currently being insufflated to the other one of the first and second pressures.

In another embodiment of the invention the pressure at which the cavity is being insufflated is altered a plurality of times during the carrying out of the procedure, and each time the pressure is altered, the pressure is altered from the one of the first and second pressures at which the cavity is currently being insufflated to the other one of the first and second pressures.

In another embodiment of the invention the pressure at which the cavity is being insufflated during the carrying out of the procedure is altered at time spaced apart intervals, and in general, at irregular time spaced apart intervals.

Preferably, the second pressure is lower than the first pressure.

Advantageously, the cavity is initially insufflated at the first pressure.

In one embodiment of the invention the procedure being carried out in the cavity comprises a suturing procedure.

Preferably, the cavity is insufflated at the first pressure during identification of a location in the cavity at which a suture is to be inserted in tissue thereof, and on each location being identified, the pressure in the cavity is altered to the second pressure to reduce tautness in the tissue to enable invaginating of the tissue in which the suture is to be inserted.

Advantageously, each time invaginating of tissue in which a suture is to be inserted has been completed, the pressure in the cavity is altered to the first pressure.

Preferably, the pressure in the cavity is maintained at the first pressure during insertion of a suture in the corresponding invaginated part of the tissue.

In another embodiment of the invention the pressure in the cavity is maintained at the second pressure until a suture has been inserted into the corresponding invaginated part of the tissue.

In one embodiment of the invention the cavity is insufflated by an insufflator configured to supply the insufflating gas to the cavity to selectively and alternately insufflate the cavity at the first pressure and the second pressure.

Preferably, the insufflator is configured to be responsive to a signal indicative of a change pressure request to alter the pressure at which the insufflating gas is being supplied to the cavity from the one of the first and second pressures at which the cavity is currently being insufflated to the other one of the first and second pressures.

In another embodiment of the invention the method further comprises producing a signal indicative of a change pressure request for reception by the insufflator each time a change in pressure in the cavity from the one of the first and second pressure at which the cavity is currently being insufflated to the other one of the first and second pressures is required.

Preferably, the signal indicative of a change pressure request is produced remotely of the insufflator.

Advantageously, the signal indicative of a change pressure request is transmitted to the insufflator by any one or more of wirelessly acoustically, or by wire.

In one embodiment of the invention the signal indicative of a change pressure request is produced by operating any one or more of a foot operated electric switch, a hand operated electric switch, a foot operated pneumatic switch or a hand operated pneumatic switch to produce one of an electrical signal or a pneumatic signal.

In another embodiment of the invention the signal indicative of a change pressure request is produced each time operation of an instrument in the cavity commences.

In one embodiment of the invention the signal indicative of a change pressure request is produced each time operation of an instrument in the cavity terminates.

In another embodiment of the invention the signal indictive of a change pressure request is produced by a detecting means provided for detecting commencement and/or termination of operation of an instrument in the cavity. Preferably, the detecting means for detecting commencement and/or termination of operation of an instrument is mounted on the instrument.

In another embodiment of the invention the signal indicative of a change pressure request is produced by a controller adapted for controlling the operation of the instrument, and/or is produced in response to operation and termination of the instrument.

In another embodiment of the invention the signal indicative of a change pressure request is produced by an electrical switch mounted on the instrument, and operable by a person using the instrument.

In another embodiment of the invention the insufflator comprises a first receiving means for receiving the signal indicative of a change pressure request.

Preferably, the first receiving means comprises any one or more of a wireless receiver, a microphone, a hardwire connector configured for connecting to a wire carrying the signal indicative of the change pressure request, or a pneumatic sensor configured for receiving a pneumatic signal indicative of a change pressure request.

In another embodiment of the invention a flow control means is provided in the insufflator for controlling the rate of flow at which the insufflating gas is supplied to the cavity for maintaining the pressure at the one of the first and second pressures at which the cavity is to be insufflated.

In one embodiment of the invention the flow control means is responsive to a signal indicative of the pressure in the cavity for maintaining the pressure in the cavity at the one of the first and second pressures at which the pressure in the cavity is to be maintained.

In another embodiment of the invention the pressure in the cavity is altered from the one of the first and second pressures at which the cavity is currently being insufflated to the other one of the first and second pressures by the flow control means in response to the signal indicative of a change pressure request.

In one embodiment of the invention a control means is provided, the control means being configured to control the operation of the flow control means in response to the signal indicative of the pressure in the cavity for maintaining the pressure in the cavity at the one of the first and second pressures at which the cavity is to be insufflated.

In another embodiment of the invention the control means is responsive to a signal indictive of a change pressure request for operating the flow control means to alter the rate at which the insufflating gas is being supplied to the cavity to alter the pressure in the cavity from the one of the first and second pressures at which the cavity is currently being insufflated to the other one of the first and second pressures.

In one embodiment of the invention a vacuum system is connected to the cavity in response to the signal indicative of a change pressure request when the pressure in the cavity is at the first pressure for rapidly reducing the pressure in the cavity from the first pressure to the second pressure.

In one embodiment of the invention the supply of insufflating gas is interrupted to the cavity when the vacuum from the vacuum system is applied to the cavity.

In another embodiment of the invention the vacuum from the vacuum system is disconnected from the cavity when the pressure in the cavity has fallen to the second pressure.

In another embodiment of the invention the delivery of insufflating gas to the cavity is reinstated on the vacuum system from the vacuum system being disconnected therefrom.

In a further embodiment of the invention the insufflating gas is supplied to the cavity at a flow rate for maintaining the pressure in the cavity at the second pressure until a signal indicative of a change pressure request is received.

In another embodiment of the invention the first receiving means is configured to produce a change signal, in response to receiving a signal indicative of a change pressure request, and the control means is responsive to the change signal for operating the flow control means to alter the flow rate at which the insufflating gas is being delivered to the cavity to alter the pressure in the cavity from the one of the first and second pressures at which the cavity is being insufflated to the other one of the first and second pressures.

Preferably, the flow control means comprises a flow controller.

In another embodiment of the invention a pressure monitoring means is provided, the pressure monitoring means being configured to monitor pressure in the cavity, and to produce a signal indicative of the pressure in the cavity.

In another embodiment of the invention the pressure value of at least one of the first and second pressures is selectable. Preferably, the first pressure is selectable from a plurality of pressure values lying in a range of 8 mmHg to 15 mmHg. Advantageously, the second pressure is selectable from a plurality of pressure values lying in the range of 0 mmHg to 8 mmHg.

Further the invention provides a method for insufflating a cavity in a human or animal subject, the method comprising:
supplying insufflating gas to the cavity from an insufflator to insufflate the cavity at either a first pressure or a second pressure,
transmitting a signal indicative of a change pressure request to the insufflator to alter the pressure at which the cavity is being insufflated from the one of the first and second pressures at which the cavity is currently being insufflated to the other one of the first and second pressures.

Preferably, the signal indicative of a change pressure request is remotely transmitted.

### Advantages of the Invention

The advantages of the invention are many. By providing the insufflator as being operable in first and second operating modes, the insufflator is particularly suitable for insufflating a cavity during an endoscopic procedure during which smoke is generated, for example, by a low pressure procedure such as a low pressure dissecting procedure, and also in the same procedure during which high pressure gas, other than insufflating gas is produced in the cavity during the procedure, or is generated in the cavity during the procedure by, for example, an argon plasma coagulation procedure. By operating the insufflator in the first operating mode, which in general would be the normal operating mode of the insufflator, the cavity pressure is maintained at the set pressure, and in the event of high pressure gases being generated in the cavity during the endoscopic procedure or being introduced into the cavity during the endoscopic procedure, such gases are readily withdrawn to reduce the cavity pressure back to the set pressure by terminating the delivery of insufflating gas to the cavity by the first control means, and operating the second control means to apply vacuum to the cavity for the first predefined time period, and if necessary, one or more second predefined time periods. Smoke generated in the cavity by, for example, a low pressure dissecting procedure, is readily removed by operating the insufflator in the second operating mode, whereby the cavity is subjected to the pressure/vacuum cycles, while at the same time maintaining the cavity pressure within plus or minus 1 mmHg or 2 mmHg about the set pressure.

By providing the insufflator with the pressure monitoring means selectively connected to the third port or the first or second outlet ports, the insufflator is suitable for use with an endoscope where only one channel is available for both insufflating the cavity and withdrawing gas from the cavity and also for monitoring the cavity pressure, and where a second channel is available in the endoscope, the cavity pressure may be monitored through the second channel by connecting the pressure monitoring means through the third port to the cavity, while insufflating of the cavity and withdrawing gases from the cavity are carried out through the first and second outlet ports, respectively, through the first channel of the cavity.

A further advantage of the invention is that by connecting the first control means and the second control means to the cavity through the instrument channel, due to the larger diameter of the instrument channel of an endoscope than the diameter of the insufflating channel thereof, significantly higher volumes of insufflating gas may be delivered to the cavity in the event of relatively high leakage from the cavity, and also relatively high volumes of gas may be withdrawn from the cavity in the event of the pressure exceeding the predefined upper pressure.

Additionally, by utilising the instrument channel to accommodate insufflating gas therethrough to the cavity being insufflated, the flow rate of insufflating gas from the insufflator to the cavity can be increased by a factor of 10 to 20, due to the fact that the instrument channel is of diameter in the range of 2.8 mm to 3.3 mm. Thus, this allows the cavity to be insufflated with insufflating gas at flow rates up to 40 litres per minute, which is more than adequate to compensate for leaks, for example, through the mouth or anus in the range of 15 litres per minute to 20 litres per minute. By virtue of the fact that the insufflator is configured to control the insufflating gas supplied therefrom at two selectable pressures, the pressure at which the cavity is insufflated can be readily and easily controlled at either of the first and second pressures, and furthermore, the pressure at which the cavity is being insufflated can be readily and easily switched from the first pressure to the second pressure and vice-versa by operating the foot pedal operated switch, or by a signal indicative of a change pressure request produced by the operation of an instrument in the cavity.

Another advantage of the insufflator is achieved by virtue of the fact that the first signal receiver is configured to receive a signal indicative of a change pressure request from the first to the second pressure or from the second to the first pressure by a voice request or by operating a foot pedal or a hand operated switch or by both a voice request and a foot pedal or a hand operated switch. This enables a change pressure request signal to be produced by the surgeon or the clinician rapidly and with minimum effort. In the case of a hand operated switch, it is envisaged that the hand operated switch may be located on the endoscope, and typically, adjacent the proximal end of the endoscope. Such a switch may be of the type which would transmit a wireless signal for reception by the first receiving means of the insufflator, for changing the pressure from the current one of the first and second pressures at which the insufflating gas is being supplied to the cavity to the other one of the first and second pressures.

A further advantage of the invention is achieved by the provision of the instrument sensor which detects the presence of an instrument or the insertion of an instrument into the instrument channel of the endoscope, which produces a signal indicative of an instrument in or being entered into the instrument channel for reception by the insufflator, which then readily switches the supply of insufflating gas to the cavity from the instrument channel to the insufflating channel of the endoscope, and on removal of the instrument from the instrument channel, in the absence of the signal indicative of an instrument in or being entered into the instrument channel, the insufflator switches the supply of insufflating gas to the cavity from the insufflating channel to the instrument channel. However, it is envisaged that in some embodiments of the invention instead of providing an instrument sensor, a manually operated switch may be provided, which typically, would be located on the endoscope adjacent the proximal end thereof which would be manually operated by a surgeon or a clinician as an instrument is being urged into the instrument channel. Such a manually operated switch would be configured to transmit a signal for reception by the second signal receiving means of the insufflator, which would then operate the insufflator to switch the delivery of the insufflating gas to the cavity from the instrument channel to the insufflating channel of the endoscope. On removal of the instrument from the instrument channel, the switch could then be again operated by a surgeon or a clinician, to transmit a further signal for reception by the second signal receiving means of the insufflator, to switch the supply of insufflating gas from the insufflating channel of the endoscope back to the instrument channel of the endoscope. Alternatively, the manually operated switch may be of the type which would continually transmit the signal until it was operated again to indicate withdrawal of the instrument from the instrument channel.

A further advantage of the invention is that by providing the instrument sensor for detecting the presence of an instrument in or being entered into the instrument channel, on receiving the signal indicative of an instrument in or being entered into the instrument channel, the insufflator also switches the pressure sensor from monitoring the pressure in the cavity through the insufflating channel to monitoring the pressure in the cavity through the instrument channel, so that the pressure in the cavity can be continuously monitored through the instrument channel.

The insufflating system according to the invention, apart from the many advantages discussed above, is also particularly suitable for use in carrying out any procedure which requires suturing in a cavity in the body of a subject being insufflated by the insufflator. By virtue of the insufflator being readily operable in response to a signal indicative of a change pressure request, the pressure in the cavity can be readily switched from one of the first and second pressures at which the cavity is being insufflated to the other one of the first and second pressures. This is a particularly important advantage during invaginating of tissue into which a suture is to be inserted, in that the pressure in the cavity can be readily reduced from the first pressure to the second pressure in order to relax the tissue in the cavity wall to enable the tissue to be significantly more easily invaginated therein. Once the tissue has been invaginated, the pressure can then be readily increased from the second pressure to the first pressure while the invaginated part is still held by the invaginating device. It has been found that by returning the pressure in the cavity from the second pressure to the first pressure, the tissue of an invaginated part while still being held by the invaginating device become tensioned, thereby reducing the force required to urge a suturing needle through the tissue of the invaginated part.

The invention will be more clearly understood from the following description of some preferred embodiments thereof which are given by way of example only with reference to the accompanying drawings.

### BRIEF DESCRIPTION OF THE DRAWINGS

FIG. 1 is a block representation of an insufflating system according to the invention comprising an insufflator also according to the invention and an endoscope also according to the invention,

FIG. 2 is a schematic cross-sectional side elevational view of the endoscope of the insufflating system of FIG. 1,

FIG. 3 is another view similar to FIG. 2 of the endoscope of FIG. 2 illustrating a portion of the endoscope in a different state to that of FIG. 2, in FIG. 4 is a partly sectional front elevational view of a subject with the endoscope of FIG. 1 inserted orally into the stomach of the subject,

FIG. 5 is a view similar to FIG. 2 of an endoscope according to another embodiment of the invention,

FIG. 6 is a perspective view of a detail of an endoscope substantially similar to the endoscope of FIG. 5 according to another embodiment of the invention,

FIG. 7 is a cross-sectional side elevational view of a detail of an endoscope substantially similar to the endoscope of FIG. 5 according to a further embodiment of the invention,

FIG. 8 is a view similar to FIG. 2 of an endoscope according to another embodiment of the invention,

FIG. 9 is a cutaway perspective view of a portion of a stomach of the subject in which an endoscopic sleeve gastroplasty procedure has been carried out using the insufflating system of FIG. 1,

FIG. 10 is a transverse cross-sectional view of the stomach of FIG. 9 also after the endoscopic sleeve gastroplasty procedure has been carried out,

FIG. 11 is a perspective view of a portion of the stomach of FIG. 9 illustrating a detail in the carrying out of the endoscopic sleeve gastroplasty procedure,

FIG. 12 is a block representation of an insufflating system according to the invention comprising an insufflator also according to the invention for insufflating a cavity in the body of a human or animal subject,

FIG. 13 is a front elevational view of a part of a human subject illustrating a part of the insufflating system extending into the stomach of the subject,

FIG. 14 is a block representation similar to that of FIG. 12 of an insufflating system according to another embodiment of the invention, and

FIG. 15 is a block representation similar to the block representation of FIG. 12 of an insufflating system according to a further embodiment of the invention.

### DETAILED DESCRIPTION OF THE INVENTION

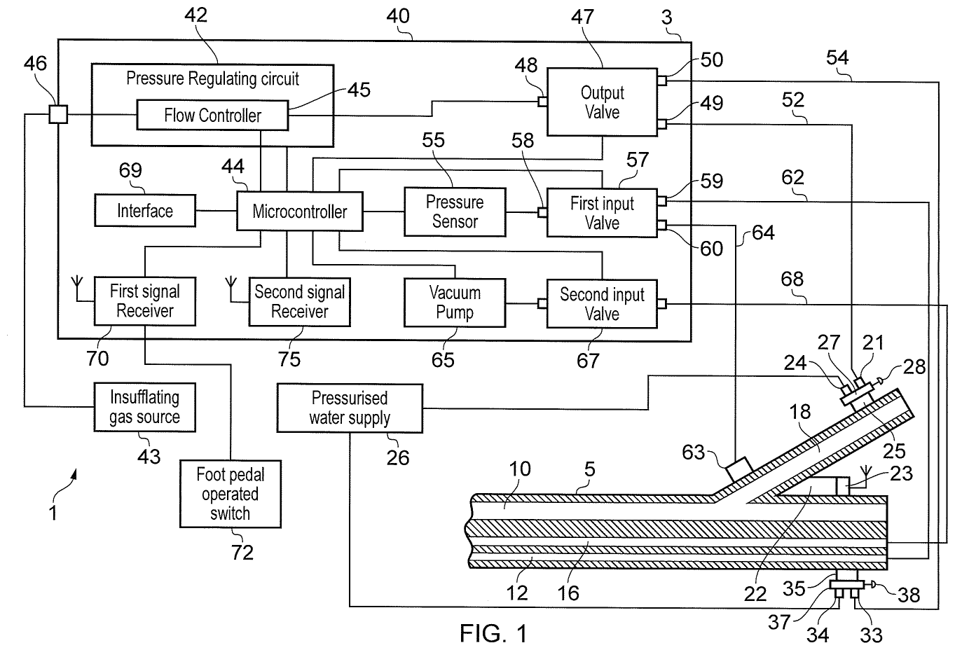

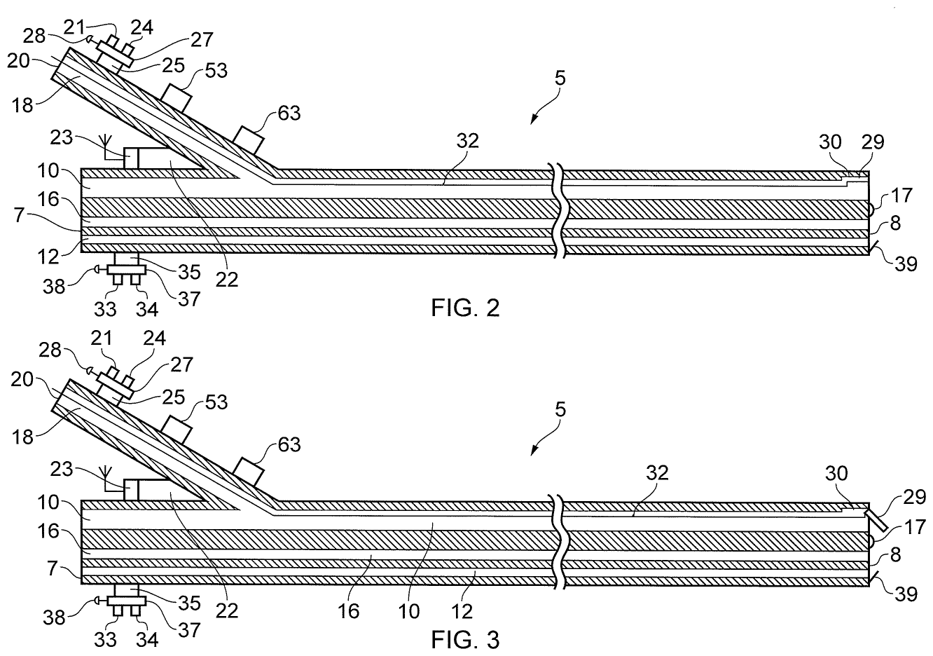

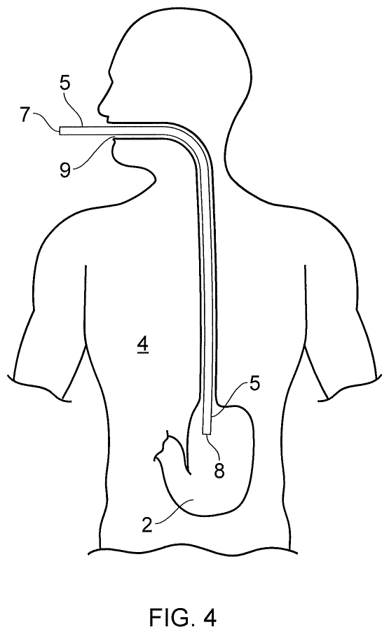

Referring to the drawings and initially to FIGS. 1 to 4 thereof, there is illustrated an insufflating system according to the invention indicated generally by the reference numeral 1 for insufflating a cavity 2, in this case the stomach in the body 4 of a human or animal subject during the carrying out of a surgical or investigative procedure in the cavity 2. The insufflating system 1 comprises an insufflator also according to the invention and indicated generally by the reference numeral 3, and an endoscope also according to the invention and indicated generally by the reference numeral 5. Only a part of the endoscope 5 is illustrated in FIG. 1, and the endoscope is illustrated schematically in FIGS. 2 and 3. Before describing the insufflating system 1 in detail, the endoscope 5 will first be described.

The endoscope 5 extends from a proximal end 7 to a distal end 8, and is of the type which is suitable for entering into a cavity 2 in the body of a human or animal subject through a natural body orifice, for example, orally through the mouth as illustrated in FIG. 4, or transanally through the rectum to a cavity. The distal end 8 of the endoscope 5 is the end which extends into the cavity 2 of the human or animal subject in which a surgical or investigative procedure is to be carried out endoscopically, with the proximal end 7 of the endoscope 5 extending externally of the subject through the mouth 9 in this case, or the rectum as the case may be. The carrying out of such procedures endoscopically will be well known to those skilled in the art.

The endoscope 5 comprises an instrument channel 10, an insufflating channel 12 and a vacuum channel 16. Provision for a light to illuminate the cavity into which the distal end 8 of the endoscope 5 extends, and an imaging system to provide vision in the interior of the cavity are included in the endoscope 5 as will be well known and understood to those skilled in the art. The instrument, insufflating and vacuum channels 10, 12 and 16 extend from the proximal end 7 of the endoscope 5 to the distal end 8 thereof. The instrument channel 10 is of a diameter suitable for accommodating instruments to the cavity, and typically, is of diameter in the range of 2.8 mm to 3.3 mm. The insufflating channel 12 is adapted for accommodating insufflating gas therethrough to the cavity, and is of diameter of approximately 1 mm. The vacuum channel 16 is configured for connecting to a vacuum system for suctioning or evacuating insufflating gas from the cavity. In embodiments of the invention in which an endoscope is provided without a dedicated vacuum channel, the instrument channel typically is selectively connectable to a vacuum system. A lens 17 of the imaging system is located adjacent the distal end 8 of the endoscope 5.

A branch channel 18 extends from the instrument channel 10 adjacent the proximal end 7 of the endoscope 5 for accommodating an instrument into the instrument channel 10. The branch channel 18 is also adapted for connecting to the insufflator 3, as will be described below, for connecting a supply of insufflating gas thereto for delivery through the instrument channel 10 for insufflating the cavity 2 when the instrument channel 10 is not being used for accommodating an instrument therethrough, and in some cases when an instrument is extending through the instrument channel 10. Appropriate seals (not shown) are provided in the branch channel 18 adjacent a proximal end 20 thereof for sealing the branch channel 18 when the instrument channel 10 is not accommodating an instrument therethrough, and also for sealably engaging an instrument in the branch channel 18 adjacent the proximal end 20 thereof when the instrument channel 10 is accommodating an instrument therethrough.

An instrument sensor 22 is located adjacent the proximal end 20 of the branch channel 18 for detecting the presence of an instrument in the branch channel 18 or in the instrument channel 10, or for detecting an instrument being entered into the branch channel 18 or the instrument channel 10 adjacent the proximal end 7 thereof. The instrument sensor 22 is configured to produce a signal indicative of an instrument in or being entered into the instrument channel, which will be described in more detail below, in response to detecting an instrument in or being entered into the instrument channel 10. The signal indicative of an instrument in or being entered into the instrument channel is produced by the instrument sensor 22 for reception by the insufflator 3. In this embodiment of the invention the instrument sensor 22 comprises a wireless transmitter 23 configured to transmit the signal in a Bluetooth protocol indicative of an instrument in or being entered into the instrument channel. The instrument sensor 22 is configured to operate the transmitter 23 to continue to produce the signal indicative of an instrument in or being entered into the instrument channel from commencement of detection of the instrument being entered into the branch channel 18 or the instrument channel 10 until the instrument is no longer detected in either the branch channel 18 or the instrument channel 10, in other words, until the instrument has been withdrawn from the endoscope.

A first inlet port 25 communicates with the branch channel 18 adjacent the proximal end 20 but distally from the seals (not shown) therein for accommodating both insufflating gas from the insufflator 3 and pressurised water from a pressurised water supply 26 into the branch channel 18, and in turn into the instrument channel 10. A manually operated first bi-state valve, in this case, a monostable bi-state first spool valve 27 selectively and alternately connects insufflating gas from the insufflator 3 and pressurised water from the pressurised water supply 26 to the inlet port 25. A first inlet port 21 of the first spool valve 27 is adapted for receiving the insufflating gas from the insufflator 3 as will be described below. A second inlet port 24 of the first spool valve 27 is connected to the pressurised water supply 26. The first spool valve 27 is stable in a normal first state with the first inlet port 21 connected through to the first inlet port 25 of the branch channel 18 and the second inlet port isolated from the branch channel 18. The first spool valve 27 is operable in an unstable second state with the first inlet port 21 thereof isolated from the first inlet port 25 of the branch channel 18 and the second inlet port 24 connected to the first inlet port 25 of the branch channel 18. A thumb operated button 28 operates the first spool valve 27 from the first state to the second state. The first spool valve 27 is operated into the second state thereof by depressing the thumb operated button 28, and is held in the second state for so long as the thumb operated button 28 is depressed. Typically, the pressured water supply 26 is derived from a pressurised container of water, and in general, the water is used for cleaning the lens 17 of the imaging system, although the pressurised water supply may be used for flushing out the cavity.

A directing means is located adjacent the distal end 8 of the endoscope 5 for directing pressurised water exiting through the instrument channel 10 adjacent the distal end thereof to the lens 17 of the imaging system. In this embodiment of the invention the directing means comprises a deflecting element, namely, a deflecting plate 29 which is located in a recess 30 extending into the endoscope 5 adjacent the distal end 8 and adjacent the instrument channel 10. The deflecting plate 29 is urgeable distally through the recess 30 from a rest state illustrated in FIG. 2 located within the recess 30 to a deflecting state illustrated in FIG. 3 with the deflecting plate 29 angled relative to the instrument channel 10 for directing water from the instrument channel 10 onto the lens 17. The deflecting plate 29 is spring biased by a spring (not shown) so that as the deflecting plate 29 is urged distally from the recess 30, the spring urges the deflecting plate 29 into the deflecting state illustrated operating in FIG. 3. An operating means, in this embodiment of the invention an elongated operating cable 32 extends from the deflecting plate 29 longitudinally through the instrument channel 10, and in turn through the branch channel 18 and outwardly from the branch channel 18 adjacent the proximal end 20 thereof in order to enable manual operation of the deflecting plate 29 between the rest state and the deflecting state.

In some embodiments of the invention it is envisaged that the operating cable 32 and the thumb operated button 28 of the first spool valve 27 may be interconnected, so that as the thumb operated button switch 28 of the first spool valve 27 is being operated to deliver the pressurised water into the instrument channel 10, the deflecting plate 29 would be simultaneously operated from the rest state to the deflecting state for directing the pressurised water onto the lens 17, and on release of the button switch 28 the deflecting plate 29 would be returned to the rest state in the recess 30.

Returning now to FIG. 1, a second inlet port 35 is located in the endoscope 5 adjacent the proximal end thereof communicating with the insufflating channel 12 to deliver insufflating gas from the insufflator 3 and pressurised water from the pressurised water supply 26 into the insufflating channel 12. A second two state valve, in this case a monostable bi-state second spool valve, similar to the first spool valve 27 selectively and alternately connects the insufflating gas from the insufflator 3 and the pressurised water from the pressurised water supply 26 to the second inlet port 35. The second spool valve 37 comprises a first inlet port 33 and a second inlet port 34. The first inlet port 33 as will be described below is connected to the insufflator 3 for receiving insufflating gas therefrom, and the second inlet port 34 is connected to the pressurised water supply 36. Like the first spool valve 27 the second spool valve 37 is stable in a normal first state with the second inlet port 34 thereof isolated from the second inlet port 35 of the endoscope, and the first inlet port 33 connected to the second inlet port 35 of the endoscope for delivering insufflating gas from the insufflator 3 to the insufflating channel 12. In a second unstable state of the second spool valve 37, the first inlet port 33 thereof is isolated from the second inlet port 35 of the endoscope 5, and the second inlet port 34 of the second spool valve 37 is connected through to the second inlet port 35 of the endoscope 5 for communicating the pressurised water supply 26 with the insufflating channel 12. A thumb operated button 38 operates the second spool valve 37 from the first state to the second state. The thumb operated button 38 is depressed for operating the second spool valve 37 from the first state to the second state, and for so long as the thumb operated button switch is depressed the second spool valve 37 is retained in the second state. A deflector plate 39 at the distal end 8 of the endoscope 5 adjacent but spaced apart from the distal end of the insufflating channel 12 deflects pressurised water from the insufflating channel 12 onto the lens 17 of the endoscope 5 as will be understood by those skilled in the art.

Turning now to the insufflator 3, the insufflator 3 is configured to supply insufflating gas to the endoscope 5 for insufflating the cavity 2, in which the procedure is being carried out, selectively and alternately at two pressures, namely, a first pressure and a second pressure. The first pressure is selectable within a range of pressure values which typically lie in the range of 8 mmHg to 15 mmHg. The second pressure is lower than the first pressure, and is selectable within a range of pressure values which typically lie in the range of 0 mmHg to 8 mmHg. The insufflator 3, as will be described in detail below, is configured to supply insufflating gas at one of the first and second pressures, to in turn insufflate the cavity 2 at that one of the first and second pressures, and on reception of a remotely generated signal indicative of a change pressure request, the insufflator 3 alters the pressure to the other one of the first and second pressures to insufflate the cavity 2 at that other one of the first and second pressures, as will be described below. The insufflator 3 has the capacity to supply the insufflating gas at both the first pressure and the second pressure at a flow rate in the range of 1 litre per minute to 30 litres per minute, and may be configured to supply the insufflating gas at both the first and second pressures at flow rates of up to 40 litres per minute.

The insufflator 3 comprises a housing 40 within which a pressure regulating means, in this case, a pressure regulating circuit 42 is located, and is operable under the control of a control means, which in this embodiment of the invention comprises a signal processor, in this case provided by a microcontroller 44, which is also located in the housing 40. It will of course be appreciated that any other suitable control means besides a microcontroller may be provided, for example, a programmable logic controller, a microprocessor or any other suitable signal processor. The pressure regulating circuit 42 is operated under the control of the microcontroller 44 for selectively and alternately supplying the insufflating gas to the cavity 2 to insufflate the cavity 2 at either the first pressure or the second pressure.

Insufflating gas is delivered to the pressure regulating circuit 42 from a pressurised source of the insufflating gas, which typically comprises carbon dioxide. The pressurised source of the insufflating gas may comprise a pressurised cylindrical container of the insufflating gas, which may be located internally in or externally of the housing 40, or alternatively, the insufflator 3 may be adapted for connecting the pressure regulating circuit 42 to a pressurised hospital supply of carbon dioxide or other suitable insufflating gas. In this embodiment of the invention the insufflator 3 is adapted to receive insufflating gas from a source 43 provided by a pressurised hospital supply of insufflating gas. The hospital supply of insufflating gas is supplied at a pressure considerably in excess of the highest selectable value of the first pressure. A main input port 46 is located in the housing 40 for connecting the pressure regulating circuit 42 to the insufflating gas source 43.

In this embodiment of the invention the pressure regulating circuit 42 comprises a flow control means, in this case a flow controller 45 through which the insufflating gas is supplied through the pressure regulating circuit 42. The flow controller 45 is operated under the control of the microcontroller 44 in response to the pressure monitored in the cavity 2 for controlling the flow rate at which the insufflating gas is supplied by the pressure regulating circuit 42, for in turn controlling the pressure at which the insufflating gas is supplied to the cavity 2 and for maintaining the pressure in the cavity 2 substantially at the one of the first and second pressures at which the cavity 2 is to be insufflated. The monitoring of the pressure in the cavity is described below.

The insufflating gas at the one of the first and second pressures, at which the insufflating gas is being supplied from the pressure regulating circuit 42, is delivered through an output valve means, in this case, an output valve, which comprises a motor controlled output valve 47 operated under the control of the microcontroller 44. The output valve 47 is located in the housing 40 and comprises an input port 48 connected to the flow controller 45 of the pressure regulating circuit 42. A pair of output ports of the output valve 47 define a first output port 49 and a second output port 50 through which insufflating gas is delivered from the insufflator 3. The output valve 47 may comprise a two state valve, a three state valve or a four state valve. If the output valve 47 comprises a two state valve, in a first one of the two states the second output port 50 is isolated from the input port 48, and the first output port is connected to the input port 48 for delivering the insufflating gas from the insufflator 3, and in a second state of the two states the first output port 49 is isolated from the input port 48, and the second output port 50 is connected to the input port 48 for delivering the insufflating gas through the second output port 50. If the output valve 47 comprises a three state valve, the first and second states thereof would be the same as the first and second states of a two state valve, and in the third state thereof both the first and second output ports 49 and 50 would be connected to the input port 48 so that insufflating gas would be simultaneously delivered through the first and second output ports 49 and 50. If the output valve 47 comprises a four state valve the first to the third state thereof would be the same as the first to the third states of the three state valve and in the fourth state thereof the first and second output ports 49 and 50 would both be isolated from the input port 48 thereby preventing delivery of insufflating gas from the insufflator 3.

In this case the output valve 47 comprises a two state valve, and under the control of the microcontroller 44 in the first state thereof insufflating gas is delivered solely through the first output port, and in the second state thereof insufflating gas is delivered solely through the second output port.

The first output port 49 is connected by a first output gas line 52 to the first inlet port 21 of the first spool valve 27 for delivering the insufflating gas into the branch channel 18, and in turn through the instrument channel 10 of the endoscope 5 to the cavity 2 when the first spool valve 27 is in the first state. The second output port 50 of the output valve 47 is connected by a second output gas line 54 to the first inlet port 33 of the second spool valve 37 for delivering the insufflating gas from the second output port 50 into the insufflating channel 12, and in turn to the cavity 2 through the second inlet port 35 when the second spool valve 37 is in the first state. The control of the output valve 47 by the microcontroller 44 for alternately delivering the insufflating gas to the branch channel 18 and the insufflating channel 12 of the endoscope 5 will be described in more detail below.

A pressure monitoring means, in this embodiment of the invention a pressure sensor 55 is located in the housing 40 for monitoring the pressure in the cavity 2. The microcontroller 44 reads signals from the pressure sensor 55, which are indicative of the pressure in the cavity 2, and operates the flow controller 45 to control the flow rate of the insufflating gas through the pressure regulating circuit 42, to in turn control the pressure in the cavity 2 at the one of the first and second pressures. Thus the flow controller 45, the microcontroller 44 and the pressure sensor 55 act as a closed feed back loop for maintaining the pressure in the cavity 2 at the one of the first and second pressures.

An input valve means, in this embodiment of the invention a solenoid operated input valve 57 located in the housing 40 comprises an output port 58, which is connected to the pressure sensor 55, and a pair of input ports, namely, a first input port 59 and a second input port 60. The first input port 59 of the input valve 57 is connected to the insufflating channel 12 of the endoscope 5 adjacent the proximal end 7 thereof by a first input gas line 62. The second input port 60 of the input valve 57 is connected to the branch channel 18 of the endoscope 5 by a second input gas line 64. The second input gas line 64 is connected to the branch channel 18 through a port 63 in the branch channel 18 adjacent the proximal end 20 thereof but distally from the seals (not shown) in the branch channel 18 adjacent the distal end thereof. The input valve 57 is operated under the control of the microcontroller 44 to selectively and alternately connect the output port 58 to the first and second input ports 59 and 60, for in turn connecting the pressure sensor 55 to the cavity 2 selectively and alternately through the insufflating channel 12 and the instrument channel 10 of the endoscope 5 for monitoring the pressure in the cavity 2.

As will be described below, when the output valve 47 is operated by the microcontroller 44 for connecting the insufflating gas to the branch channel 18 of the endoscope 5 for insufflating the cavity 2 through the instrument channel 10, the input valve 57 is operated by the microcontroller 44 for connecting the pressure sensor 55 to the insufflating channel 12 of the endoscope 5 for monitoring the pressure in the cavity 2 through the insufflating channel 12. When the output valve 47 is operated by the microcontroller 44 for delivering the insufflating gas to the cavity 2 through the insufflating channel 12 of the endoscope 5, the input valve 57 is operated by the microcontroller 44 for connecting the pressure sensor 55 to the instrument channel 10 for monitoring the pressure in the cavity 2 through the instrument channel 10. This enables the pressure sensor 55 to continuously monitor the pressure in the cavity selectively and alternately through either the insufflating channel 12 or the instrument channel 10.

However, in some embodiments of the invention it is envisaged that two pressure sensors will be provided, one of which would communicate with the insufflating channel 12 for monitoring the pressure in the cavity 2 through the insufflating channel 12, and the other pressure sensor would communicate with the branch channel 18 for monitoring the pressure in the cavity 2 through the instrument channel 10. In which case, the input valve 57 would be omitted, and the microcontroller 44 would determine from which of the pressure sensors the cavity pressure should be read, and would read the cavity pressure accordingly from the appropriate one of the pressure sensors. It is also envisaged that the one or two pressure sensors may be located in the endoscope, and in which case signals indicative of the pressure monitored by the pressure sensor or sensors would be transmitted to the microcontroller 44 of the insufflator 3 either wirelessly or by wire.

A vacuum system, in this case comprising a vacuum pump 65 located in the housing 40 is operated under the control of the microcontroller 44 for producing a vacuum. A vacuum control valve means, in this embodiment of the invention a solenoid operated vacuum control valve 67 is located in the housing 40 for connecting the vacuum pump 65 to the vacuum channel 18 of the endoscope 5 adjacent the proximal end 7 thereof through a vacuum line 68. The vacuum pump 65 and the vacuum control valve 67 are operated under the control of the microcontroller 44 for applying a vacuum to the vacuum channel 18, and in turn to the cavity 2 for drawing insufflating gas from the cavity 2 for rapidly reducing the pressure in the cavity 2 from the first pressure to the second pressure in response to the signal indicative of a change pressure request, as will be described below, when the microcontroller 44 is operating the pressure regulating circuit 42 to insufflate the cavity 2 at the first pressure. When the insufflating gas is being supplied by the pressure regulating circuit 42 at the first pressure, the microcontroller in response to the signal indicative of a change pressure request operates the flow controller 45 to terminate supply of insufflating gas to the cavity 2, and activates the vacuum pump 65 and operates the vacuum control valve 67 into the open state to apply a vacuum to the cavity to rapidly reduce the pressure in the cavity to the second pressure. On the pressure in the cavity 2 being reduced to the second pressure, the microcontroller 44 deactivates the vacuum pump 65 and operates the vacuum control valve 67 into the closed state and operates the flow controller 45 to again supply the insufflating gas at a suitable flow rate to maintain the pressure in the cavity 2 at the second pressure.

In the event of an endoscope 5 not having a dedicated vacuum channel, the vacuum control valve 67 would be configured to connect the vacuum pump to the instrument channel 10 either to the instrument channel 10 adjacent the proximal end 7 thereof or to the branch channel 18, and more typically, to a port on the branch channel 18 similar to the port 63, which would be located adjacent the proximal end 20 of the branch channel 18 but distally of the seals (not shown) therein adjacent the proximal end 20. In some embodiments of the invention the vacuum pump may be dispensed with, and the insufflator 3 would be connected to a vacuum system of a hospital, and in which case the vacuum system of the hospital would be applied to the vacuum line through the vacuum control valve 67 of the insufflator 3.

An interface means provided by an interface 69, which may comprise a touch screen, a keypad, a voice recognition module or other suitable interface, is located on the housing 40 and is connected to the microcontroller 44 for inputting data to the microcontroller 44, and in particular, for inputting selected values of the first and second pressures at which the pressure of the insufflating gas is selectively and alternately controlled by the pressure regulating circuit 42.

A first receiving means, in this embodiment of the invention a first signal receiver 70 is located in the housing 40 for receiving a remotely generated signal indicative of a change pressure request to change the pressure from the one of the first and second pressures at which the insufflating gas is currently being supplied to the cavity 2 by the pressure regulating circuit 42 to the other one of the first and second pressures. The first signal receiver 70 may be of the type to receive a hardwired remotely generated signal indicative of a change pressure request, or it may be configured to receive a remote wirelessly transmitted signal indicative of a change pressure request, or it may be configured with a voice recognition module for receiving a voice generated change pressure request, or it may be configured to receive the change pressure request inputted through a touch screen or other such tactile input means on the housing 40, or through a touch screen of the interface 69. Indeed, in some embodiments of the invention the first signal receiver 70 may comprise one or more of a hardwire system for receiving a remotely generated signal indicative of a change pressure request, a remotely transmitted wireless signal indicative of a change pressure request or a voice generated change pressure request, or a touch screen or other tactile input means on the housing 40.

The first signal receiver 70 is configured to produce an electronic change signal in response to receiving a signal indicative of a change pressure request. The change signal produced by the first signal receiver is applied to the microcontroller 44. On the microcontroller 44 receiving the change signal from the first signal receiver 70, the microcontroller 44 operates the flow controller 49 of the pressure regulating circuit 42 to alter the flow rate at which the insufflating gas is being supplied by the pressure regulating circuit 42, for in turn altering the pressure at which the cavity 2 is currently being insufflated from the current one of the first and second pressures to the other one of the first and second pressures.

A remote signal generator for generating the remote signal indicative of a change pressure request, in this case comprises a foot pedal operated switch 72 located externally of the housing 40. The foot pedal operated switch 72 is hardwired connected to the first signal receiver 70. The foot pedal operated switch 72 is configured for generating the remote signal indicative of a change pressure request. The foot pedal operated switch 72 comprises a normally open mono-stable electric switch (not shown). By depressing the foot pedal thereof the electric switch is operated from the normally open state to the closed state to generate an electrical signal indicative of the change pressure request. The electrical signal indicative of the change pressure request is applied to the first signal receiver 70, which in response thereto produces the change signal, which is applied to the microcontroller 44.

It is envisaged that in some embodiments of the invention the remote signal generator for generating the remote signal indicative of the change pressure request may comprise a hand operated switch, which would comprise a normally open mono-stable electric switch, which when operated by hand from the normally open state to the closed state would produce the remote signal indicative of a change pressure request.

It is also envisaged that the first signal receiver 70 may be configured to receive a remotely transmitted signal indicative of a change pressure request, typically, a wirelessly transmitted signal produced as a result of operating an instrument in the cavity 2. For example, as will be described in more detail below, during suturing in a cavity, a surgeon may require to reduce the tautness in the wall of the cavity in order to permit grasping and invaginating of tissue at a location of the cavity wall to insert a suture therein. Thus, as a mechanical grasper, a helical retractor or a suctioning instrument is about to be operated to invaginate tissue at the relevant location of the cavity wall, a signal indicative of a change pressure request would be transmitted by the mechanical grasper, the helical retractor or the suctioning instrument. On reception of the signal indicative of a change pressure request, the insufflator 3 would reduce the pressure in the cavity from the first pressure to the second pressure. On completion of invaginating of the tissue at the relevant location, another signal indicative of a change pressure request would be transmitted by the mechanical grasper, the helical retractor or the suctioning instrument, to in turn return the pressure in the cavity to the first pressure. Such signals from an instrument, may be generated by a control system operating the instrument, or by a monitoring device mounted on the instrument which would monitor the operation of the instrument and transmit the signal indicative of a change pressure request at or before the commencement and on or after termination of the particular task being carried out by the instrument for reception by the first signal receiver 70. Alternatively, a button operated signal transmitter may be provided on the instrument adjacent the proximal end thereof or on the endoscope 5 adjacent the proximal end thereof, which would be operable by a surgeon or clinician to produce the signal indicative of a change pressure request at the commencement and termination of the task during which the pressure in the cavity is to be altered from the first pressure to the second pressure or vice-versa.

A second receiving means, in this embodiment of the invention a second signal receiver 75 is located in the housing 40 for receiving the signal from the instrument sensor 22 of the endoscope 5, which is indicative of an instrument in or being entered into the instrument channel. In this embodiment of the invention the second signal receiver 75 comprises a wireless receiver configured to operate in a Bluetooth protocol. On receiving the signal from the instrument sensor 22 indicative of an instrument in or being entered into the instrument channel, the second signal receiver 75 produces an electric signal corresponding to the signal indicative of an instrument in or being entered into the instrument channel to the microcontroller 44. On receiving the corresponding signal, the microcontroller 44 operates the output valve 47 to switch the insufflating gas from the first output port 49 to the second output port 50, and operates the input valve 57 to switch the pressure sensor 55 from the first input port 59 to the second input port 60, so that the insufflating gas is delivered to the cavity 2 through the insufflating channel 12 of the endoscope 5 and the pressure in the cavity 2 is monitored by the pressure sensor 55 through the instrument channel 10.

When the instrument has been withdrawn from the instrument channel 10 and the branch channel 18, the instrument sensor 22 ceases to transmit the signal indicative of an instrument in or being entered into the instrument channel. On detecting termination of the signal indicative of an instrument in or being entered into the instrument channel, the second signal receiver 75 outputs a signal indicative of an instrument being withdrawn from the instrument channel to the microcontroller 44, and the microcontroller 44 on receiving the signal indicative of an instrument being withdrawn from the instrument channel operates the output valve 47 to switch the insufflating gas from the second output port 50 to the first output port 49, and operates the input valve 57 to switch the pressure sensor 55 from the second input port 60 to the first input port 59, so that the insufflating gas is again delivered to the cavity 2 through the instrument channel 10, and the pressure in the cavity 2 is monitored by the pressure sensor 55 through the insufflating channel 12 of the endoscope 5. Accordingly, for so long as there is no instrument in the instrument channel 10, the cavity 2 is insufflated through the instrument channel 10 and the pressure in the cavity is monitored through the insufflating channel 12, and vice-versa when an instrument is in the instrument channel 10.

For a better understanding of the insufflating system 1, the use of the insufflating system 1 will now be described. With the insufflator 3 connected to the endoscope 5 through the gas lines 52, 54, 62, 64 and 68 and with the first and second inlet ports 25 and 35 of the endoscope connected to the pressurised water supply 26 through the first and second spool valves 27 and 37, and with the insufflator 3 connected to the insufflating gas source 43, the insufflating system 1 is ready for use. The surgeon or clinician selects and enters the values of the first and second pressures to which the cavity 2 is to be insufflated through the interface 69 to the microcontroller 44, which are stored in memory of the microcontroller 44. The endoscope 5 if not already inserted orally into the cavity 2, in this case the stomach in the body 4 of the subject, in which the procedure is to be carried out, the endoscope 5 is entered into the cavity 2 orally. The insufflator 3 is then operated to deliver the insufflating gas.

In the normal operating mode of the insufflator 3, the pressure regulating circuit 42 is operated under the control of the microcontroller 44 to control the pressure at which the insufflating gas is supplied to the cavity 2 by the insufflator 3 at the first pressure. On activation of the insufflator 3, the microcontroller 44 is programmed to operate the flow controller 45 of the pressure regulating circuit 42, in response to the pressure in the cavity 2 read by the microcontroller 44 from the pressure sensor 55, to control the flow rate of the insufflating gas through the pressure regulating circuit 42, to in turn control the pressure of the insufflating gas to insufflate the cavity 2 at the selected first pressure. Since initially, in general, there will be no instruments in the instrument channel 10 of the endoscope 5, no signal will be transmitted by the instrument sensor 22, and the microcontroller 44 controls the output valve 47 and the input valve 57, so that insufflating gas is supplied to the cavity 2 through the instrument channel 10 and the pressure in the cavity 2 is monitored by the pressure sensor 55 through the insufflating channel 12. Since insufflating gas is not being delivered through the insufflating channel 12, the pressure in the cavity 2 is continuously monitored through the insufflating channel 12.

The microcontroller 44 controls the flow controller 45 to control the rate at which the insufflating gas is being delivered through the output valve 47 to the cavity 2 in which the procedure is being carried out, in response to the cavity pressure read by the pressure sensor 55 for maintaining the cavity pressure substantially at the one of the first and second pressures at which the insufflating gas is to be supplied by the insufflator 3, which initially will be at the first pressure.

On the surgeon or a clinician wishing to reduce the pressure in the cavity 2 from the first pressure to the second pressure, in order to, for example, reduce the tautness in the wall of the cavity in which the procedure is being carried out, for example, to slacken the tension in the cavity wall to facilitate invaginating tissue of the cavity wall by a surgical mechanical grasper, a helical retractor or a suction instrument to facilitate suturing of the cavity wall, the surgeon or clinician operates the foot pedal operated switch 72 to generate the signal indicative of a change pressure request. On receiving the signal indicative of a change pressure request, the first signal receiver 70 produces the change signal which is read by the microcontroller 44. The microcontroller 44 in response to the change signal operates the flow controller 45 of the pressure regulating circuit 42 to temporarily terminate delivery of the insufflating gas to the cavity 2. Simultaneously, the microcontroller 44 activates the vacuum pump 65, and operates the vacuum control valve 67 from the closed state to the open state to draw insufflating gas from the cavity 2 through the vacuum channel 18, and through the vacuum line 68. The microcontroller 44 reads the signals indicative of the pressure in the cavity 2 from the pressure sensor 55, and on the cavity pressure being reduced to the second pressure, the microcontroller 44 operates the vacuum control valve 67 from the open state into the closed state and deactivates the vacuum pump 65. Simultaneously, the microcontroller 44 operates the flow controller 45 of the pressure regulating circuit 42 to recommence delivery of the insufflating gas to the cavity 2, and controls the flow controller 45 in response to the pressure in the cavity 2 read from the pressure sensor 55 to control the pressure of the insufflating gas to maintain the pressure in the cavity 2 at the second pressure.

The microcontroller 44 continues to operate the flow controller 45 of the pressure regulating circuit 42 to continue to maintain the pressure of the insufflating gas in the cavity 2 at the second pressure in response to the pressure in the cavity 2 read from the pressure sensor 55 until the next signal indicative of a change pressure request is received by the first signal receiver 70.

It is however envisaged that in cases where the leakage of the insufflating gas from the cavity 2 is relatively high, when the pressure of the insufflating gas is to be reduced from the first pressure to the second pressure, it may not be necessary to activate the vacuum pump to draw insufflating gas from the cavity, since due to a high leakage rate of insufflating gas from the cavity 2, the pressure in the cavity 2 may fall sufficiently rapidly on the flow controller 45 of the pressure regulating circuit 42 being operated by the microcontroller 44 to temporarily terminate delivery of the insufflating gas to the cavity 2. In which case, on the pressure in the cavity 2 falling to the second pressure, the flow controller 45 would again be operated under the control of the microcontroller 44 to maintain the pressure in the cavity 2 at the second pressure.

On the surgeon or clinician wishing to return the pressure at which the insufflating gas is being supplied by the insufflator 3 to the cavity 2 from the second pressure to the first pressure, the surgeon or clinician operates the foot pedal operated switch 72 to generate the signal indicative of a change pressure request as already described. On receiving the signal indicative of a change pressure request, the first signal receiver 70 again outputs a change signal to the microcontroller 44. The microcontroller 44 in turn operates the flow controller 45 of the pressure regulating circuit 42 to increase the flow rate at which the insufflating gas is being delivered to the cavity 2 to increase the pressure at which the cavity 2 is being insufflated from the second pressure to the first pressure. The insufflator 3 continues to operate as described, and each time the first signal receiver 70 receives a signal indicative of a change pressure request, it produces a change signal, which in turn results in the microcontroller 44 operating the flow controller 45 of the pressure regulating circuit 42 and/or the vacuum pump 65 and the vacuum control valve 67, as the case may be, to alter the pressure at which the cavity is being insufflated from the current one of the first and second pressures to the other one of the first and second pressures, and so the insufflator 3 continues to operate.

As discussed above if the remote signal generator instead of being provided by a foot pedal operated switch, were provided by a hand operated electrical switch, the operation of the hand operated electrical switch would be similar to that described with reference to a foot pedal operated switch. The hand operated switch would be operated to produce a signal indicative of a change pressure request, which would be received by the first signal receiver 70, which would in turn output the change signal. The microcontroller 44 on receiving the change signal would operate the flow controller 45 of the pressure regulating circuit 42 and/or the vacuum pump 65 and the vacuum control valve 67, as the case may be, to alter the pressure at which the insufflating gas is being supplied to the cavity 2 from the current one of the first and second pressures to the other one of the first and second pressures.

Alternatively, if the insufflator 3 were supplying the insufflating gas at the first pressure and if the signal indicative of a change pressure request was produced in response to the commencement of operation of an instrument in the cavity, the first signal receiver 70, on receipt of the signal indicative of a change pressure request would produce a change signal, and the microcontroller 44 in response thereto would operate the flow controller 45 of the pressure regulating circuit 42 to temporarily terminate the supply of insufflating gas to the cavity and simultaneously would activate the vacuum pump 65 and operate the vacuum control valve 67 into the open state to reduce the pressure at which the cavity 2 is being insufflated from the first pressure to the second pressure. On termination of the operation of the instrument in the cavity 2, a signal indicative of a change pressure request would again be transmitted, which would be received by the first signal receiver 70, which in turn would produce the change signal. The microcontroller 44 in response to the change signal would operate the flow controller 45 of the pressure regulating circuit 42 to increase the flow rate of the insufflating gas, to in turn increase the pressure at which the cavity 2 is being insufflated from the second pressure to the first pressure, which in general would be the first pressure.

Until an instrument is entered into the instrument channel 10 of the endoscope 5, the output valve 47 is operated by the microcontroller 44 in the first state with the insufflating gas being delivered to the cavity 2 through the first output port 49 thereof, and in turn through the first output gas line 52 and the instrument channel 10, and the input valve 57 is operated by the microcontroller 44 in the first state with the pressure in the cavity 2 being continuously monitored by the pressure sensor 55 through the insufflating channel 12, the first input gas line 62 and the first input port 59.

On the instrument sensor 22 of the endoscope 5 detecting an instrument being entered into or in the branch channel 18 or the instrument channel 10, the instrument sensor 22 produces the signal indicative of an instrument in or being entered into the instrument channel. The second signal receiver 75, on receiving the signal indicative of an instrument in or being entered into the instrument channel produces the signal corresponding to the signal indicative of an instrument in or being entered into the instrument channel. The microcontroller 44 on receiving the signal corresponding to the signal indicative of an instrument in or being entered into the instrument channel operates the output valve 47 from the first state to the second state to in turn switch the insufflating gas from the first output port 49 to the second output port 50 thereof, and operates the input valve 57 from the first state to the second state thereof to switch the pressure sensor 55 from the first input port 59 to the second input port 60, so that the insufflating gas is delivered to the cavity 2 through the insufflating channel 12 of the endoscope 5, and the pressure in the cavity 2 is monitored by the pressure sensor 55 through the instrument channel 10.

Alternatively, instead of operating the input valve 57 from the first state to the second state to switch the pressure sensor 55 from the first input port 59 to the second input port 60, the input valve 57 may be left in the first state with the pressure sensor 55 connected through the insufflating channel 12 to the cavity 2, and in which case the pressure in the cavity 2 would be monitored by the pressure sensor 55 through the insufflating channel 12 at time spaced apart predefined time periods during which insufflating of the cavity 2 through the insufflating channel 12 would be paused for monitoring of the pressure in the cavity 2. Typically, each predefined time period during which the pressure is monitored in the cavity 2 by the pressure sensor 55 will be in the order of one to two seconds, and the pressure in the cavity will be monitored at intervals of approximately one second. In general, it is envisaged that the microcontroller 44 would operate the flow controller 45 or a valve (not shown) located between the flow controller 45 and the output valve 47 to pause insufflating of the cavity during each predefined time period in order to permit monitoring of the pressure within the cavity. Alternatively, if the output valve 47 were a four state valve, as described above, the output valve 47 would be operated in the fourth state to isolate the insufflating gas from the insufflating channel 12 during each predefined time period during which the pressure in the cavity is being monitored by the pressure sensor 55.

The microcontroller 44 continues to operate the output valve 47 in the first state with the insufflating gas being delivered to the cavity through the insufflating channel 12 and the input valve 57 in either the first or second states, as the case may be, with the pressure in the cavity being monitored by the pressure sensor 55 either continually through the instrument channel 10 or intermittently through the insufflating channel 12 until the instrument has been removed from the instrument channel 10. At that stage the instrument sensor 22 ceases to produce the signal indicative of an instrument in or being entered into the instrument channel. On the second signal receiver 75 detecting termination of the signal indicative of an instrument in or being entered into the instrument channel, the second signal receiver 75 produces a signal indicative of an instrument being withdrawn from an instrument channel to be read by the microcontroller 44. The microcontroller 44 on receiving the signal indicative of an instrument being withdrawn from an instrument channel operates the output valve 47 from the second state to the first state to switch the insufflating gas from the second output port 50 to the first output port 49 to recommence insufflating of the cavity 2 through the instrument channel 10, and operates the input valve 57 from the second state to the first state to switch the pressure sensor 55 from the second input port 60 to the first input port 59, so that the pressure in the cavity 2 is monitored through the insufflating channel 12.

When the insufflator 3 is delivering the insufflating gas through the instrument channel 10, and it is required to clean the lens 20, the first spool valve 27 is operated from the first state to the second state by the thumb operated button 28 for connecting the second inlet port 24 thereof to the first inlet port 25 of the branch channel 18 to deliver pressurised water from the pressurised water supply 26 to the branch channel 18, and in turn through the instrument channel 10. As the first spool valve 27 is being operated into the second state, the operating cable 32 is operated to urge the deflecting plate 29 from the rest state in the recess 30, illustrated in FIG. 2, to the deflecting state illustrated in FIG. 3 for directing the pressurised water exiting from the distal end 8 of the instrument channel 10 onto the lens 17 for cleaning thereof. On the other hand, when the output valve 47 is being operated in the second state to deliver the insufflating gas to the cavity 2 through the insufflating channel 12, should it be desired to clean the lens 17 of the endoscope 5, the second spool valve 37 is operated by the thumb operated button 38 thereof from the first state to the second state to connect the pressurised water supply 26 from the second inlet port 34 thereof to the second inlet port 35 of the endoscope 5, to in turn deliver the pressurised water through the insufflating channel 12, and in turn to the lens 20 for cleaning thereof. A fixed deflector plate 39 at the distal end 8 of the endoscope 5 adjacent the distal end of the insufflating channel 12 deflects the pressurised water from the insufflating channel 12 to the lens 17.

It is envisaged that in embodiments of the invention where the output valve 47 is provided as a three state valve or a four state valve, when an instrument is located in the instrument channel 10 of the endoscope 5, and the instrument is of diameter or transverse cross-section such that the instrument channel could accommodate insufflating gas therethrough, instead of operating the output valve 47 from the first state to the second state in response to a signal indicative of an instrument in or being entered into the instrument channel received from the instrument sensor 22, the output valve 47 could be operated from the first state to the third state with insufflating gas being delivered through both the first and second outlet ports 49 and 50. This would thus allow the cavity to be insufflated through both the insufflating channel 12 and the instrument channel 10, and would be particularly advantageous in the event of the leakage of insufflating gas from the cavity being relatively high.

However, in the case of the cavity being insufflated through the insufflating channel 12 and the instrument channel 10, the pressure in the cavity could be monitored either through the insufflating channel 12 or the instrument channel 10. However, since the volume of insufflating gas being delivered through the instrument channel 10 would most likely be less than the volume of insufflating gas being delivered through the insufflating channel 12, it would be preferable to monitor the pressure in the cavity through the instrument channel 10, since the need to interrupt the insufflating gas for the predefined pressure monitoring time periods during monitoring of the pressure in the cavity would have less effect on the insufflating of the cavity by monitoring the pressure in the cavity through the instrument channel 10 rather than through the insufflating channel 12.

To operate the output valve 47 in the third state, in embodiments of the invention where the output valve 47 is provided as a three state or a four state valve, would require a further additional signal to be produced for reception by the insufflator 3, so that the microcontroller 44 would operate the output valve 47 in the third state on receiving the additional signal. Such an additional signal may be produced by any suitable means, for example, by an additional foot pedal operated switch which could be operated remotely of the insufflator by the surgeon or clinician, or by a hand operated switch, or by a voice signal, or by any other suitable means. A suitable receiver for receiving the additional signal could be provided in the insufflator, for example, a third signal receiver or either the first or second signal receivers could be adapted to receive the additional signal to operate the output valve in the third state, and on receiving the signal, the signal receiver would produce an appropriate signal to the microcontroller 44 to operate the output valve in the third state.

It is also envisaged that in embodiments of the invention in which the output valve 47 is provided as a four state valve, instead of operating the flow controller 45 of the pressure regulating circuit 42 to temporarily terminate delivery of insufflating gas from the insufflator 3 to the cavity 2, the output valve 47 could be operated in the fourth state to isolate the first and second output ports 49 and 50 from the pressure regulating circuit 42, to thereby interrupt delivery of insufflating gas to the cavity. In which case, the microcontroller would be suitably programmed so that when the flow controller 45 is operating to supply the insufflating gas at the first pressure, on receiving a change pressure request, the microcontroller would operate the output valve 47 in the fourth state, and simultaneously activate the vacuum pump 65 and operate the vacuum control valve 67 in the open state to draw insufflating gas from the cavity until the pressure in the cavity would be reduced to the second pressure. At which stage, the microcontroller would deactivate the vacuum pump and operate the vacuum control valve 67 into the closed state, and simultaneously operate the output valve from the fourth state to the state in which it had been operating prior to be operated into the fourth state.

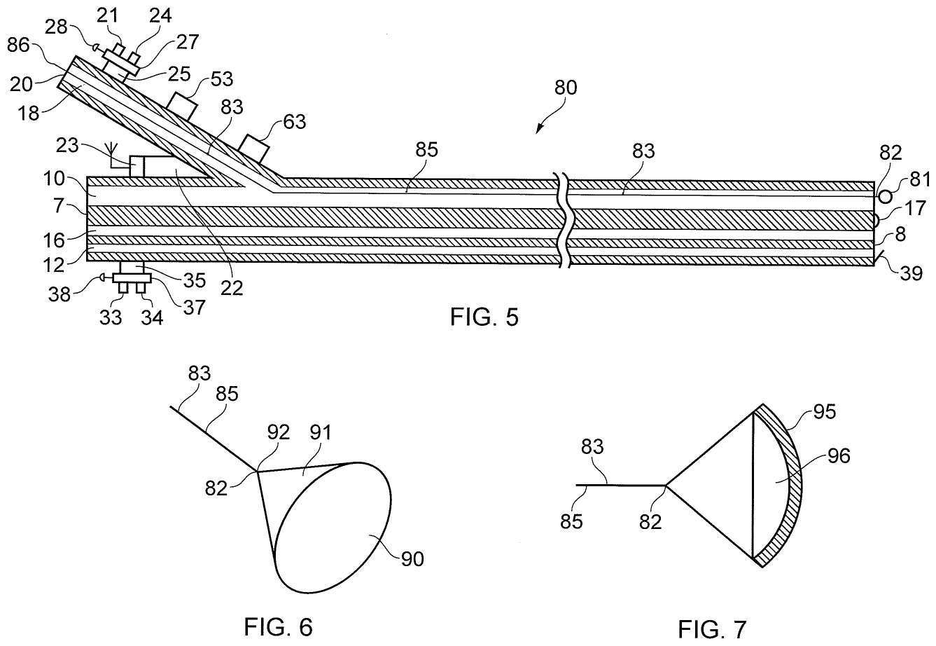

Referring now to FIG. 5 there is illustrated an endoscope according to another embodiment of the invention indicated generally by the reference numeral 80. The endoscope 80 is substantially similar to the endoscope 5, and similar components are identified by the same reference numerals. The only difference between the endoscope 80 and the endoscope 5 is that in this embodiment of the invention the directing means for directing pressurised water onto the lens 17, instead of being provided by a deflecting plate located in the endoscope, is provided by a separate directing means, which is urgeable through the instrument channel 10 each time the pressurised water is being delivered through the instrument channel 10 for cleaning the lens 17 of the imaging system. In this embodiment of the invention the directing means is provided by a deflecting element comprising a spherical element 81 connected to a distal end 82 of an elongated operating member 83. The operating member comprises a semi-rigid cable 85 extending from the distal end 82 to a proximal end 86. The spherical element 81 is urgeable through the instrument channel 10 by the operating member 83 to a position just distally from the distal end 8 of the instrument channel 10, so that when pressurised water impinges on the spherical element 81, the pressurised water is deflected from the spherical element 81 onto the lens 17 for cleaning thereof. On completion of delivery of the pressurised water into the instrument channel 10, the spherical element 81 is withdrawn through the instrument channel 10 by the operating member 83.

Otherwise, the endoscope 80 is similar to the endoscope 5 and its use is likewise similar, and may also be connected to the insufflator 3.

Referring now to FIG. 6 there is illustrated an alternative directing means for use with the endoscope 80. The directing means in this case is connected to the distal end 82 of an operating member 83, only a distal portion of which is illustrated. The operating member 83 is similar to the operating member 83 described with reference to the endoscope 80. In this embodiment of the invention the directing means comprises a conical element 90 defining a conical surface 91 terminating in a proximal apex 92. The conical element 90 is connected to the distal end 82 of the operating member 83 adjacent the apex 92 thereof, so that when the conical element 90 is located just distally from the distal end 8 of the instrument channel 10 of the endoscope 80, the apex 92 thereof faces proximally. Therefore, the pressurised water exiting the instrument channel 10 adjacent the distal end 8 thereof impinges on the conical surface 91 of the conical element 90, and is deflected from the conical surface 91 onto the lens 17 for cleaning thereof. Referring now to FIG. 7 there is illustrated another alternative directing means for directing pressurised water from the distal end 8 of the instrument channel 10 to the lens 17 of the endoscope 80 for cleaning thereof. In this embodiment of the invention the directing means comprises a deflecting element 95 of dish shaped construction. The deflecting element 95 is connected to the distal end 82 of the operating member 83 so that when viewed through the instrument channel 10, the deflecting element 95 presents a concave deflecting surface 96. Thus, when the deflecting element 95 is located just distally of the distal end 8 of the instrument channel 10, and pressurised water is delivered through the instrument channel 10, the pressurised water impinges on the concave surface 96 of the deflecting element and is deflected onto the lens 17 for cleaning thereof.

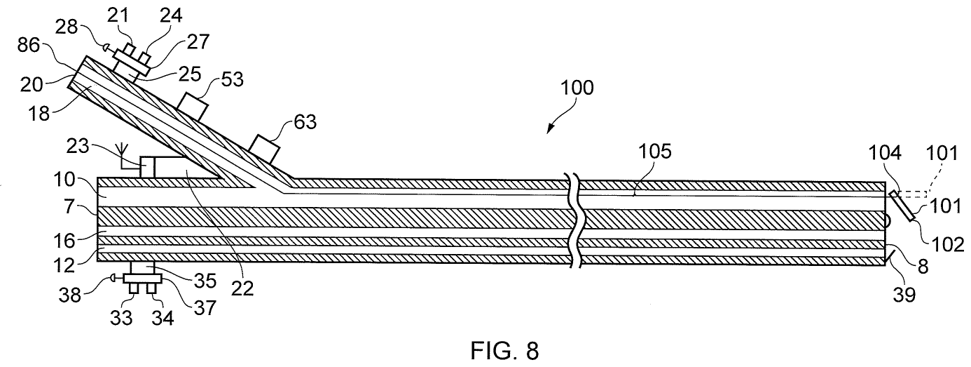

Referring now to FIG. 8 there is illustrated an endoscope according to another embodiment of the invention indicated generally by the reference numeral 100. The endoscope 100 is similar to the endoscope 80 of FIG. 5, and similar components are identified by the same reference numerals. The only difference between the endoscope 100 of FIG. 8 and the endoscope 80 of FIG. 5 is in the directing means for directing pressurised water as it exits through the distal end 8 of the instrument channel 10 onto the lens 17 for cleaning thereof. In this embodiment of the invention the directing means comprises a directing element 101 comprising a deflecting plate 102 which is coupled to the distal end 104 of an operating member 105. The operating member 105 is of length to extend the length of the instrument channel 10 from the proximal end 20 of the branch channel 18 to the distal end 8 of the instrument channel 10, and to extend proximally from the proximal end 20 of the branch channel 18 for operating thereof. In this embodiment of the invention the deflecting plate 102 is resiliently coupled to the operating member 105, typically, by a torsion spring (not shown), and is urgeable from a rest state illustrated in broken lines in FIG. 8 extending substantially in-line with the operating member 105 to a deflecting state illustrated in full lines in FIG. 8 angled relative to the operating member 105 for deflecting pressurised water exiting the distal end 8 of the instrument channel 10 onto the lens 17 for cleaning thereof. The deflecting plate 102 is retained in the rest state by the instrument channel 10 while it is being urged through the instrument channel 10. On exiting the instrument channel 10 adjacent the distal end 8 thereof, the torsion spring (not shown) biasing of the deflecting plate 102 urges the deflecting plate 102 from the rest state to the deflecting state. Thus, when it is desired to clean the lens 17, and the cavity in which the procedure is being carried out is being insufflated through the instrument channel 10, the deflecting plate 102 is urged through the instrument channel 10 in the rest state by the operating member 105 until the deflecting plate 102 exits the distal end 8 of the instrument channel 10 and springs into the deflecting state. The first spool valve 27 is then operated for delivering the pressurised water into the instrument channel 10, and the deflecting plate 102 now in the deflecting state, deflects the pressurised water as it exits the distal end 8 of the instrument channel 10 onto the lens 17 for cleaning thereof.

On completion of cleaning of the lens 17, the deflecting plate 102 is withdrawn from the instrument channel 10 by the operating member 105.

Otherwise, the endoscope 100 and its operation is similar to the endoscope 80 described with reference to FIG. 5.

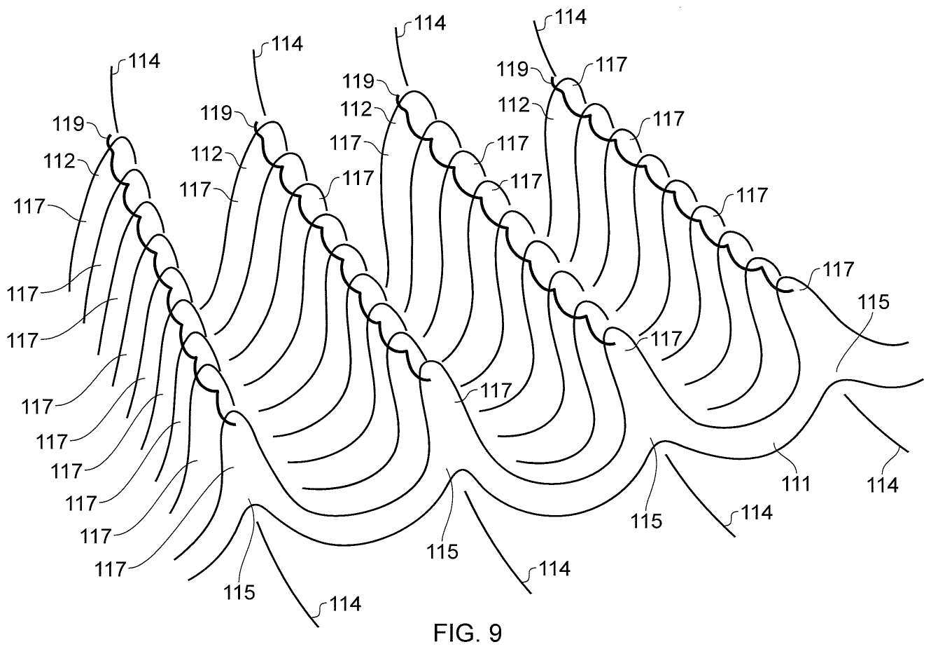

Referring now to FIGS. 4 and 9 to 11 the use of the insufflating system 1 comprising the insufflator 3 and the endoscope 5 will now be described in the carrying out of a procedure in a cavity in the body of a human or animal subject. In this embodiment of the invention the procedure is being carried out in the stomach 2 of the subject and the procedure is an endoscopic sleeve gastroplasty procedure, whereby the volume of the stomach 2 is reduced by effectively forming a gastric sleeve 110 extending through the stomach 2. The gastric sleeve 110 is formed by gathering the stomach wall 111 at longitudinally spaced apart sections 112, typically, from five to nine sections spaced apart along the length of the stomach 2. In FIG. 9 a part of each of four such gathered sections 112 is illustrated, and in FIG. 10 which illustrates a transverse cross-sectional view of the stomach 2, one of the gathered sections 112 is illustrated. At each section 112, the stomach wall 111 is gathered together along a transversely extending arcuate line 114 in the lower half of the stomach. At a plurality of spaced apart locations 115 along the transverse arcuate line 114 of each section 112, tissue of the stomach wall 111 is invaginated to form a plurality of spaced apart invaginated parts 117 at the locations 115 along the arcuate line 114. The invaginated parts 117 along each transverse arcuate line are then drawn tightly together by a suture 119 to form the gathered section 112. With the invaginated parts 117 along the arcuate line 114 of each section 112 gathered tightly together, the gastric sleeve 110 is complete.

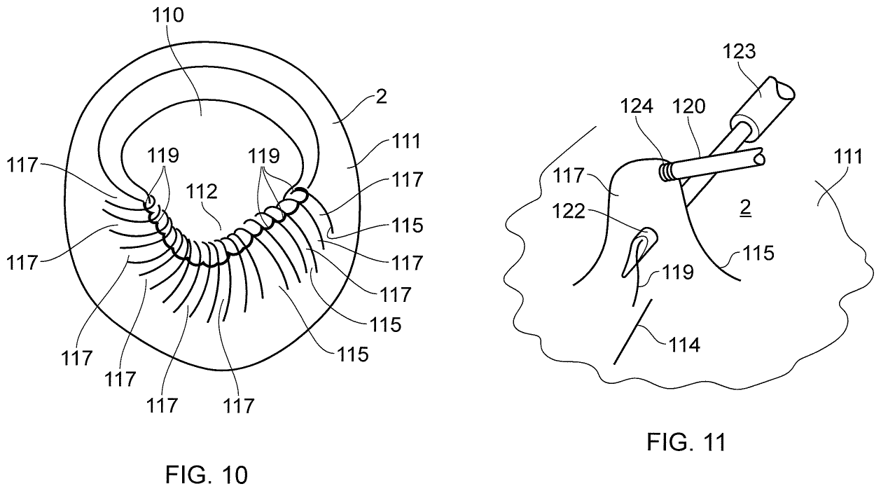

The tissue at each location 115 is invaginated by a helical retractor 120. On each invaginated part 117 having been invaginated by the helical retractor 120, the suture 119 is inserted into the invaginated part 117 by a suturing needle 122 of a suturing instrument 123. A helix 124 of the helical retractor 120 is inserted into the tissue at each location 115 at which the invaginated part 117 is to be formed, and the portion of the tissue into which the helix 124 of the helical retractor 120 is inserted is pulled away from the stomach wall 111 by the helical retractor 120 to form the invaginated part 117 of the tissue at the location 115. The invaginated part 117 is held invaginated by the helical retractor 120 while the suture 119 is being inserted through the invaginated part 117 by the suturing needle 122, see FIG. 11.

The helical retractor 120 and the suturing instrument 123 may be incorporated into a single endoscopic sleeve gastroplasty instrument, such as, for example the endoscopic sleeve gastroplasty instrument sold under the Trade Mark OVERSTITCH by Apollo Endosurgery, or the helical retractor 120 and the suturing instrument 123 may be provided as two separate instruments. If the helical retractor 120 and the suturing instrument 123 are provided as a single instrument, the instrument is passed into the stomach 2 through the instrument channel 10 of the endoscope 5. On the other hand, if the helical retractor 120 and the suturing instrument 123 are provided as two separate instruments, typically, the suturing instrument 123 is inserted through the instrument channel 10 of the endoscope 5 and the helical retractor 120 may be inserted through the vacuum channel 16 of the endoscope 5, or through a separate tube extending along the endoscope 5 from the proximal end 7 to the distal end 8, and releasably secured to the endoscope 5 by clips of the type disclosed in PCT Published Application Specification No. WO 2021/209983, or via a second instrument channel, which is provided in some commercially available endoscopes, and which also may be provided in the endoscope 5.

To carry out the procedure, the endoscope 5 is inserted orally into the stomach 2 of the subject as illustrated in FIG. 4. The insufflator is connected to the endoscope 5 as already described with reference to FIG. 1. The first and second output ports 49 and 50 of the output valve 47 are connected to the branch channel 18 and the insufflating channel 12 of the endoscope 5 through the gas lines 52 and 54, respectively. The first and second input ports 59 and 60 of the input valve 57 are connected to the insufflating channel 12 and the branch channel 18 of the endoscope 5 through the gas lines 62 and 64, respectively. The vacuum control valve 67 is connected to the vacuum channel 16 of the endoscope 5 through the vacuum line 68. With the insufflator 3 connected to the endoscope 5 of the insufflating system 1 and the insufflator 3 connected to the insufflating gas source 43, a surgeon on clinician selects and enters the desired values of the first and second pressures to which the stomach 2 is to be insufflated during the procedure through the interface 69, and the insufflating system 1 is ready for use.

Since initially there are no instruments in the instrument channel 10, the insufflator 1 is operated initially to insufflate the stomach 2 at the first pressure through the instrument channel 10, and the pressure in the stomach 2 is monitored through the insufflating channel 12 by the pressure sensor 55. In this embodiment of the invention the suturing instrument 123 and the helical retractor 120 are separate instruments. The suturing instrument 123 is entered into the stomach 2 through the instrument channel 10 and the helical retractor 120 is entered into the stomach 2 through the separate tube (not shown) attached to the endoscope 5.

On the entry of the suturing instrument 123 into the instrument channel 10 being detected in the branch channel 18 by the instrument sensor 22 of the endoscope 5, the instrument sensor 22 transmits the signal indicative of an instrument in or being entered into the instrument channel. On the signal indicative of an instrument in or being entered into the instrument channel 10 being received by the second signal receiver 75, the second signal receiver 75 outputs the signal corresponding to the signal indicative of an instrument in or being entered into the instrument channel to the microcontroller 44, which in turn operates the output valve 47 and the input valve 57 from the first states thereof to the second states thereof, to in turn switch insufflating of the stomach 2 from the instrument channel 10 to the insufflating channel 12, and to switch monitoring of the pressure in the stomach 2 by the pressure sensor 55 from the insufflating channel 12 to the instrument channel 10.

The helix 124 of the helical retractor 120 is urged to the first of the locations 115 at which the tissue is to be invaginated to form the first invaginated part 117. In order to reduce the tension in the stomach wall 111 to allow the tissue at the location 115 to be invaginated, the surgeon operates the foot pedal switch 72 which produces the signal indicative of a change pressure request. The first signal receiver 70 on receiving the signal indicative of a change pressure request outputs the change signal to the microcontroller 44 which operates the flow controller 45 of the pressure regulating circuit 42 to terminate delivery of insufflating gas to the stomach 2, and simultaneously activates the vacuum pump 65 and operates the vacuum control valve 67 into the open state to apply a vacuum to the stomach 2 in order to rapidly reduce the pressure in the stomach from the first pressure to the second pressure. On the microcontroller 44 determining from the signal read from the pressure sensor 55 indicative of the pressure in the stomach 2 having fallen to the second pressure, the microcontroller 44 deactivates the vacuum pump 65 and operates the vacuum control valve 67 into the closed state, and simultaneously operates the flow controller 45 to maintain the pressure in the stomach 2 at the second pressure. The insufflator 3 maintains the pressure in the stomach at the second pressure during the forming of the invaginated part 117 at the location 115. On the invaginated part 117 being formed and being held invaginated by the helical retractor 120, the suturing needle 122 is inserted through the invaginated part 117 to insert the suture 119 through the invaginated part 117.

In some embodiments of the invention while the invaginated part 117 is held invaginated by the helical retractor 120, immediately after initial engagement of the suturing needle 122 with the invaginated part 117, but before urging of the suturing needle 122 through the invaginated part 117, the surgeon may operate the foot pedal switch 72 to produce the signal indicative of a change pressure request, which on being received by the first signal receiver 70 would output the change signal to the microcontroller 44. The microcontroller 44 would operate the flow controller 145 to increase the flow rate of insufflating gas to raise the pressure in the stomach 2 from the second pressure to the first pressure. Increasing the pressure in the stomach 2 from the second pressure to the first pressure while the invaginated part 117 is still held by the helical retractor 120 results in stretching of the invaginated tissue. Accordingly, increasing the pressure in the stomach from the second pressure to the first pressure while the invaginated part 117 is still held by the helical retractor 120 allows the suturing needle 122 to be urged through the stretched invaginated tissue with less force than would otherwise be required.

Once the suture has been inserted into the first of the invaginated parts 117, if the pressure in the stomach 2 has not already been raised from the second pressure to the first pressure, the surgeon operates the foot pedal operated switch 72 to produce the signal indicative of a change pressure request, which on being received by the first signal receiver 70 outputs the change signal to the microcontroller 44. The microcontroller 44 on receiving the change signal controls the flow controller 45 to increase the flow rate of insufflating gas to the stomach 2 to in turn raise the pressure in the stomach 2 from the second pressure to the third pressure. On identifying the next location 117 along the arcuate line 114 in which the tissue is to be invaginated, the helix 124 of the helical retractor 120 is inserted into the tissue adjacent the location 115. The surgeon then operates the foot pedal operated switch 72 to produce the signal indicative of a change pressure request, which on being received by the first signal receiver 70 outputs the change signal to the microcontroller 44. The microcontroller 44 as already described operates the insufflator 3 to reduce the pressure in the stomach 2 from the first pressure to the second pressure, and so the formation of the invaginated parts 117 at the locations 115 along the arcuate line 114 of the stomach wall 111 continues until all the invaginated parts 117 on that arcuate line 114 have been invaginated with the suture 119 extending therethrough.

When the formation of all the invaginated parts 117 along the arcuate line 114 has been completed and the suture 119 has been entered through the invaginated parts 117, if the pressure in the stomach 2 is at the first pressure, the surgeon again operates the foot pedal operated switch 72 to produce the signal indicative of a change pressure request, which on being received by the first signal receiver 70 outputs the change signal to the microcontroller 44. The microcontroller 44 as already described operates the flow controller to terminate delivery of insufflating gas to the stomach 2, and simultaneously activates the vacuum pump 65 to rapidly reduce the pressure in the stomach 2 from the first pressure to the second pressure. The suture 119 is then tightened to draw the invaginated parts 117 along the arcuate line 114 together to form the corresponding gathered section 112 of the stomach wall 111 and so the procedure continues until the stomach wall has been gathered along the arcuate lines 114 of each of the sections 112.

In some embodiments of the invention as each invaginated part 117 is formed it is tightly sutured to the adjacent invaginated part 117 along the corresponding arcuate line 114. When the suture 119 has been inserted through the just formed invaginated part 117, the insufflator 3 is operated as described above to reduce the pressure in the stomach 2 to the second pressure if the pressure in the stomach 2 is not already at the second pressure to facilitate drawing the just formed invaginated part 117 to its adjacent invaginated part 117 by the suture 119.

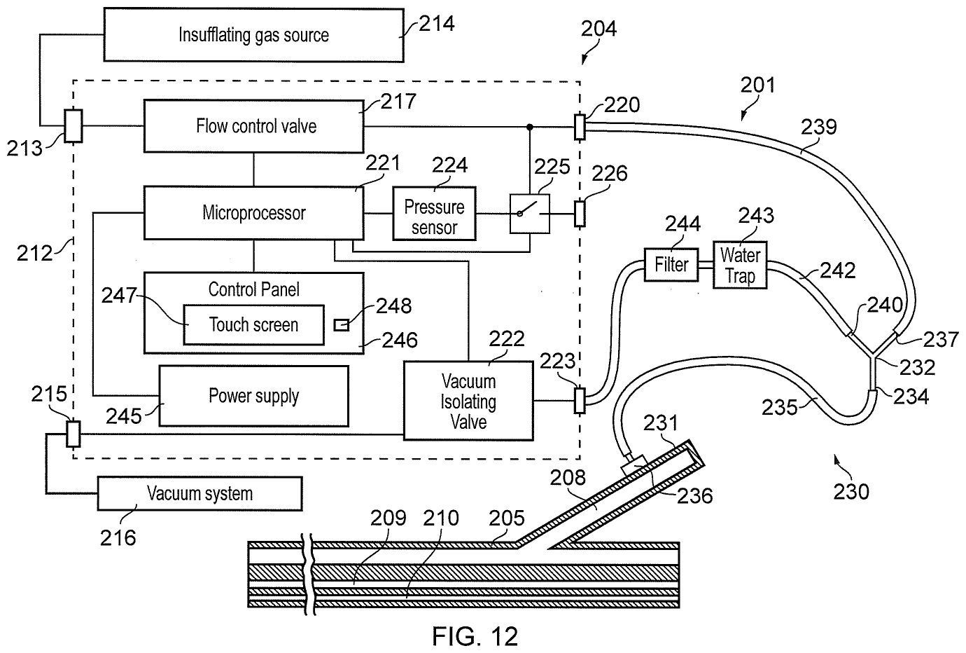

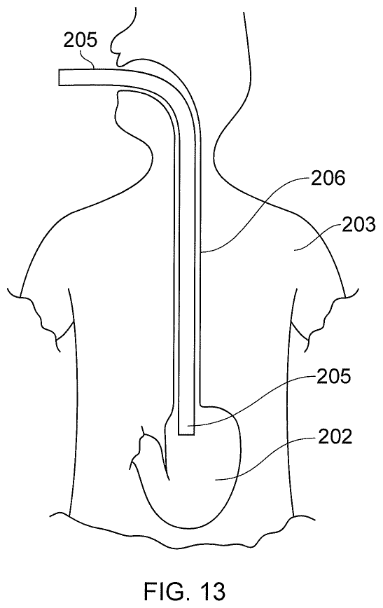

Referring now to FIGS. 12 and 13 thereof, there is illustrated an insufflating system also according to the invention indicated generally by the reference numeral 201 for insufflating a cavity in the body of a human or animal subject. In this embodiment of the invention the insufflating system 201 will be described for insufflating a stomach 202 of a human subject 203.

The insufflating system 201 comprises an insufflator also according to the invention and indicated generally by the reference numeral 204 and an endoscope 205 connected to the insufflator 204. In this embodiment of the invention the endoscope 205 is entered orally through the oesophagus 206 into the stomach 202 of the subject 203. The endoscope 205 comprises a channel capable of supplying an insufflating gas to the stomach 202 of the subject 203, which in this embodiment of the invention comprises an instrument channel 208 of the endoscope 205, although, it will be appreciated that the channel capable of supplying insufflating gas to the stomach 202 may be any other suitable channel of the endoscope 205, for example, an insufflating channel 209 of the endoscope 205, a vacuum channel 210 of the endoscope 205 or may be a channel defined by an elongated tube releasably attached externally to the endoscope 205 and extending along the endoscope 205, which typically, would be used for insufflating the stomach 202. Such a tube having a channel extending therethrough for releasably attaching to an endoscope is disclosed in PCT Published Application Specification No. WO 2021/209983 of the present applicant.

The insufflator 204 as will be described in detail below is selectively and alternately operable in two operating modes, namely, a first operating mode and a second operating mode. In the first operating mode the insufflator 204 is configured to insufflate the stomach at a set pressure, and in the event of a high pressure gas, other than the insufflating gas, being generated in the stomach 202 or introduced into the stomach 202 during a procedure being carried out in the stomach 202, to automatically withdraw the high pressure gas, and any smoke which may be generated in the stomach 202 as a result of, for example, an argon plasma coagulation submucosal dissection procedure. In the second operating mode, the insufflator 204 is configured to insufflate the stomach 202 and to withdraw smoke generated in the stomach 202 during a low pressure dissection or coagulating procedure.

In the first operating mode the insufflator 204 insufflates the stomach 202 to maintain the pressure in the stomach 202, namely, the cavity pressure at the set pressure, which is selectable, and in this case is selectable in a range from 2 mmHg to 20 mmHg, and on the pressure in the stomach 202 exceeding a predefined upper pressure, for example, as a result of an argon plasma procedure, the insufflator automatically applies a vacuum to the stomach 202 to withdraw the high pressure gas and smoke, and to reduce the pressure in the stomach 202 to or below the set pressure. In this embodiment of the invention the predefined upper pressure is set at 1 mmHg above the set pressure, although the predefined upper pressure may be in the range of 0.5 mmHg to 5 mmHg above the set pressure. In the second operating mode, the insufflator 204 is configured to sequentially and alternately apply insufflating gas to the stomach 202 and apply a vacuum to the stomach 202 in continuous sequential pressure/vacuum cycles to maintain an intermittent flow of insufflating gas through the stomach 202 to withdraw smoke generated by the low pressure procedure, while at the same time maintaining the stomach 202 insufflated at the set pressure plus or minus 1 mmHg or 2 mmHg above the set pressure.

However, while the insufflating system 201 will be described for insufflating the stomach 202 in a human subject 203, it will be readily apparent to those skilled in the art that the insufflating system 201 may be used for insufflating any cavity in the body of a human or an animal subject.

Before describing the endoscope 205 and the connection of the insufflator 204 to the endoscope 205, the insufflator 204 will first be described.

The insufflator 204 comprises a housing 212. A first inlet port 213 is provided in the housing 212 for connecting the insufflator 204 to an external supply of pressurised insufflating gas, typically, a pressurised source of insufflating gas, and generally, a pressurised insufflating gas source 214 of carbon dioxide of the type commonly available in an operating theatre of a hospital. A second inlet port 215 is located in the housing 212 for connecting the insufflator 204 to a vacuum source, in this case a vacuum system 216 of the type commonly available in an operating theatre of a hospital.

A first control means for controlling the supply and the rate of the supply at which the insufflating gas is supplied from the insufflator 204 to the stomach 202 of the subject 203 comprises a flow control valve 217. The flow control valve 217 is located in the housing 212 and is connected between the first inlet port 213, and a first outlet port 220 located in the housing 212. The flow control valve 217 is operated under the control of a signal processor for controlling the supply and the rate of supply of the insufflating gas through the first outlet port 220 from the insufflator 204 to the stomach 202 for maintaining the pressure in the stomach 202 of the subject 203 at the set pressure. The signal processor, in this case comprises a microprocessor 221, although any other suitable signal processor may be used, for example, a microcontroller, a programmable logic controller or any other suitable signal processor.

A second control means for controlling application of a vacuum to the stomach 202 from the vacuum system 216 comprises an isolating valve 222 located in the housing 212 between the second inlet port 215, and a second outlet port 223 located in the housing 212. The isolating valve 222 is operable under the control of the microprocessor 221 between an isolating state with the vacuum system 216 isolated from the second outlet port 223, and a communicating state for selectively applying the vacuum from the vacuum system 216 to the second outlet port 223 and in turn to the stomach 202 of the subject 203, for in turn drawing smoke and argon gas from the stomach 202 and for reducing the pressure in the stomach 202 to the set pressure, as will be described in detail below.

A pressure monitoring means for monitoring pressure in the stomach 202 during insufflating thereof comprises a pressure sensor 224 located in the housing 212 for producing a signal indicative of the stomach pressure, which is read by the microprocessor 221. A solenoid operated two-way valve 225 operable under the control of the signal processor 221 selectively connects the pressure sensor 224 to the first outlet port 220 or to a third port 226, so that the stomach pressure may be monitored through either the first outlet port 220 or through the third port 226. The stomach pressure is monitored through the third port 226 if there is an additional available channel in the endoscope 205 other than the instrument channel 208 to allow the stomach pressure to be monitored continuously. In this embodiment of the invention neither the insufflating channel 209 or the vacuum channel 210 of the endoscope 205 are available to provide continuous monitoring of the stomach pressure, and the stomach pressure is monitored through the first outlet port 220.

The microprocessor 221 is programmed to read the signal produced by the pressure sensor 224 at predefined time intervals of approximately 1.5 seconds, and at the end of each predefined time interval, the microprocessor 221 operates the flow control valve 217 or the isolating valve 222 to pause the supply of insufflating gas to the stomach 202 or to pause the application of vacuum to the stomach 202, as the case may be, for a predefined monitoring time period of 0.5 seconds in order to allow the pressure at the pressure sensor 224 to equalise with the pressure in the stomach 202. At the end of each monitoring time period, the microprocessor 221 reads the signal from the pressure sensor 224, which is indicative of the stomach pressure. However, it is envisaged that the predefined intervals may be of time duration in the range of 1 second to 3 seconds, and the predefined monitoring time periods may be of time duration of 0.25 seconds to 0.75 seconds. On the other hand, if the third port 226 were connected directly through to the stomach 202 through another channel of the endoscope, the microprocessor 221 would be programmed to read the signal from the pressure sensor 224 at predefined time intervals of milliseconds duration, typically, of 10 milliseconds duration.

A connecting means comprising a tube set 230 connects the instrument channel 208 of the endoscope 205 adjacent a proximal end 231 thereof to the insufflator 204. The tube set 230 comprises a Y-connector 232 having a first port 234 connected by a first conduit 235 to the instrument channel 208 of the endoscope 205 through a port 236 of the endoscope 205 adjacent the proximal end 231 of the instrument channel 208. A second port 237 of the Y-connector 232 is connected through a second conduit 239 to the first outlet port 220 of the insufflator 204. A third port 240 of the Y-connector 232 is connected by a third conduit 242 to the second outlet port 223 of the insufflator 204 through a water collecting means, namely, a water trap 243 and a filter 244. Accordingly, the first outlet port 220 and the second outlet port 223 are connected through the tube set 230 to the instrument channel 208 of the endoscope 205 for sequentially and alternately supplying insufflating gas and applying a vacuum to the stomach 202 through the instrument channel 208 of the endoscope 205, as will be described below.

The filter 244 is provided for capturing bacteria, viruses, smoke and other matter drawn from the stomach 202, and in this embodiment of the invention comprises a bacterial-viral filter with a second activated carbon filter layer. The water trap 243 is provided for collecting water entrained in the gases drawn from the stomach 202, and protects the filter 244.

The microprocessor 221 as discussed above reads the signal from the pressure sensor 224 which is indicative of the stomach pressure, and in response to the read signal, the microprocessor 221 controls the operation of the flow control valve 217 for controlling the supply and the flow rate of insufflating gas to the stomach 202, and for selectively isolating the insufflating gas from the stomach 202. The microprocessor 221 also controls the isolating valve 222 in response to the signal read from the pressure sensor 224 for applying vacuum to the stomach 202, in the event of the stomach pressure exceeding the predefined upper pressure, as will be described below.

A power supply 245 located in the housing 212 powers the microprocessor 221 and the other components in the insufflator 204.

An interface, which may comprise a touch screen, a keypad, a voice recognition interface or any other suitable interface, is located in a control panel 246 in the housing 212 for inputting data, to the microprocessor 221, namely, the selected set pressure at which the stomach 202 is to be maintained during insufflating thereof, and other data necessary for the operation of the insufflator 204. In this case, the interface comprises a touch screen 247. The touch screen 247 also displays data relating to the insufflating of the stomach 202, such as the stomach pressure and other relevant data. A membrane pushbutton operated switch 248 is also located in the control panel 246 in the housing 212 for selecting and switching between the two operating modes of the insufflator 204.

The microprocessor 221 is programmed to operate the insufflator 204 in the two operating modes. In the first operating mode of the insufflator 204, the microprocessor 221 is programmed to operate the flow control valve 217 for supplying insufflating gas to the stomach 202 in order to maintain the stomach pressure at the set pressure. Initially on commencement of insufflating of the stomach 202, the microprocessor 221 operates the isolating valve 222 into the isolating state to isolate the second outlet port 223 from the vacuum system 216, and operates the flow control valve 217 to supply insufflating gas through the first outlet port 220, and in turn through the instrument channel 208 of the endoscope 205 to the stomach 202 for insufflating thereof. At the predefined time intervals of 1.5 seconds, the microprocessor 221 operates the flow control valve 217 to pause insufflating of the stomach 202 for the predefined monitoring time period of 0.5 seconds for monitoring the pressure in the stomach 202. At the end of each predefined monitoring time period, the microprocessor 221 reads the signal indicative of the stomach pressure from the pressure sensor 224. On the signal read from the pressure sensor 224 being indicative of the stomach pressure being below the set pressure, the microprocessor 221 is programmed to operate the flow control valve 217 to increase the flow rate of insufflating gas to the stomach 202 in order to return the stomach 202 to the set pressure and to maintain the stomach pressure at the set pressure.

If however, the signal read from the pressure sensor 224 is indicative of the stomach pressure having exceeded the predefined upper pressure, for example, as a result of an argon plasma coagulation being employed in the procedure being carried out in the stomach 202, the microprocessor 221 is programmed to operate the flow control valve 217 to isolate the insufflating gas from the first outlet port 220 and to simultaneously operate the isolating valve 222 from the isolating state to the communicating state for applying vacuum to the second outlet port 223 for a first predefined time period of approximately 3 seconds, for in turn applying vacuum to the stomach 202 in order to draw the high pressure argon gas from the stomach 202 and to reduce the stomach pressure to the set pressure. At the end of the first predefined time period, the microprocessor 221 operates the isolating valve 222 into the isolating state and reads the signal from the pressure sensor 224.

If the signal read from the pressure sensor 224 at the end of the first predefined time period is indicative of the stomach pressure having fallen to or below the set pressure, the microprocessor 221 operates the flow control valve 217 to reinstate the supply of insufflating gas to the stomach 202 to maintain the pressure in the stomach 202 at the preset pressure as already described, and leaves the isolating valve 222 in the isolating state.

However, if the signal read from the pressure sensor 224 at the end of the first predefined time period is not indicative of the stomach pressure having fallen to or below the set pressure, the microprocessor 221 is programmed to operate the isolating valve 222 again into the communicating state for at least one second time period to again apply the vacuum to the stomach 202. At the end of the first one of the second time periods, the microprocessor 221 operates the isolating valve 222 into the isolating state and reads the signal from the pressure sensor 224. If at the end of the first one of the second time periods the stomach pressure has fallen to or below the set pressure, the microprocessor 221 is programmed to operate the flow control valve 217 to reinstate the supply of insufflating gas to the stomach 202 to maintain the stomach pressure at the preset pressure as already described, and the microprocessor 221 leaves the isolating valve 222 in the isolating state.

However, if the signal read from the pressure sensor 224 at the end of the first one of the second time periods is not indicative of the stomach pressure having fallen to or below the set pressure, the signal processor 221 is programmed to again operate the isolating valve 222 into the communicating state to again apply the vacuum to the second outlet port 223, and in turn to the stomach 202 for another one of the second time periods, and so on until the pressure in the stomach 202 has fallen to or below the set pressure.

In this embodiment of the invention the first predefined time period and the second time period are of the same time duration, and are each of approximately 3 seconds, but may lie in the range of 1 second to 5 seconds. In other embodiments of the invention the second time periods may be of shorter time duration than the time duration of the first predefined time period, and in which case the time duration of the second time periods may be the same or the time durations thereof may be different, and in general, would decrease progressively.

In another embodiment of the invention the time duration of the first one of the second time periods is computed by the microprocessor 221 as a function of the difference between the set pressure and the stomach pressure read at the end of the first predefined time period, and the rate at which the stomach pressure dropped during the first predefined time period, so that at the end of the first one of the second time periods, the stomach pressure should fall to the set pressure. The duration of each one of the second and subsequent ones of second time periods is computed by the microprocessor 221 as a function of the difference between the set pressure and the stomach pressure at the end of the immediately preceding second time period, and the rate at which the stomach pressure fell during the immediately preceding second time period, so that at the end of the second time period just to be commenced, the stomach pressure should fall to the set pressure.

On the other hand, if an additional channel were available in the endoscope 205 for monitoring pressure in the stomach 202, the third port 226 would be connected through a conduit to that additional channel of the endoscope, and the microprocessor 221 would read the signal indicative of the pressure in the stomach 202 from the pressure sensor 224 at predefined time intervals, typically, of 10 milliseconds. Otherwise, operation of the insufflator 204 would be similar to that just described.

In the second operating mode of the insufflator 204, the microprocessor 221 is programmed to operate the insufflator 204 for sequentially and alternately supplying insufflating gas and applying a vacuum to the stomach 202 in sequential pressure/vacuum cycles of duration of approximately 10 seconds per cycle, in order to provide an intermittent flow of insufflating gas through the stomach 202, to draw smoke and low pressure gases introduced or generated in the stomach 202 from the stomach 202. During each pressure/vacuum cycle, the microprocessor 221 is programmed to operate the flow control valve 217 to supply insufflating gas to the stomach 202 for a predefined gas supply time period, and at the end of the predefined gas supply time period, to operate the isolating valve 222 into the communicating state for a predefined vacuum application time period to apply vacuum to the second outlet port 223, and in turn to the stomach 202. Each predefined gas supply time period is of approximately 7 seconds, and each predefined vacuum application time period is of approximately 3 seconds. During each predefined gas supply time period, the isolating valve 222 is operated in the isolating state to prevent vacuum being applied to the stomach 202, and during each predefined vacuum application time period, the flow control valve 217 is operated to isolate the first outlet port 220 from the insufflating gas source 214 to prevent insufflating gas being delivered to the stomach 202.

The alternating of the supply of insufflating gas and the application of the vacuum to the stomach 202 during each pressure/vacuum cycle maintains a flow of insufflating gas through the stomach to remove any smoke generated in the stomach 202 during, for example, a low pressure dissection procedure being carried out therein, and also to remove any low pressure gases introduced to or generated in the stomach 202 during the procedure, while at the same time the pressure in the stomach is maintained at the set pressure plus or minus 1 mmHg or 2 mmHg about the set pressure.

In use, the insufflator 204 is connected to the insufflating gas source 214 and to the vacuum system 216. The endoscope 205 is inserted orally through the oesophagus 206 into the stomach 202 of the subject 203, and the insufflator 204 is connected to the endoscope 205. The first outlet port 220 and the second outlet port 223 are connected to the instrument channel 208 of the endoscope 205 through the tube set 230. The set pressure at which the stomach 202 is to be insufflated is entered into the microprocessor 221 through the touch screen 247. In the absence of a conduit being connected to the third port 226 for connecting the third port 226 to a channel in the endoscope 205, other than the instrument channel 208, the microprocessor 221 operates the valve 225 to connect the pressure sensor 224 to the first outlet port 220. Alternatively, if the two-way valve 225 instead of being provided as a solenoid operated valve operated under the control of the microprocessor, were provided as a manually operated valve, the two-way valve would be manually operated to connect the pressure sensor 224 to the first outlet port 220. The operating mode in which the insufflator 204 is to be operated is selected by operating the membrane button operated switch 248 in the control panel 246 in order to select the operating mode in which the insufflator 204 is to operate. If an operating mode is not selected, the insufflator 204 defaults to the first operating mode. In general, it is envisaged that the first operating mode will normally be selected to commence insufflating of the stomach 202. Once the first operating mode is selected, or in default, the microprocessor 221 is programmed to operate the insufflator 204 in the first operating mode as already described and, the microprocessor 221 continues to operate the insufflator 204 in the first operating mode during the carrying out of the procedure in the stomach 202, or until the second operating mode is selected by operating the membrane button operated switch 248. On the second operating mode being selected, the microprocessor 221 is programmed to operate the insufflator 204 in the second operating mode as already described until the procedure has been completed, or until the first operating mode is again selected by operating the membrane button operated switch 248.

If the insufflator 204 is to be operated initially in the second operating mode, the second operating mode is selected by operating the membrane button operated switch 248, and the insufflator 204 continues to operate in the second operating mode until the procedure has been completed or the first operating mode is selected by operating the membrane button operated switch 248.

In general, it is envisaged that the insufflator 204 will be operated in the first operating mode in procedures where a high pressure gas, other than the insufflating gas, is likely to be introduced into the stomach 202 being insufflated or is likely to be generated in the stomach 202 during the carrying out of the procedure and also where smoke is likely to be generated in the cavity during a high pressure dissection or coagulation process. The insufflator 204, in general, will also be operated in the first mode of operation where it is unlikely that a high pressure gas, other than the insufflating gas, will be introduced to the stomach 202 or generated therein during the procedure. However, the second operating mode of the insufflator 204 will be selected prior to or just prior to the commencement of a procedure during which smoke will be generated in, for example, a low pressure dissecting or coagulation procedure for withdrawing the smoke while maintaining the stomach pressure at the set pressure plus or minus 1 mmHg or 2 mmHg about the set pressure.

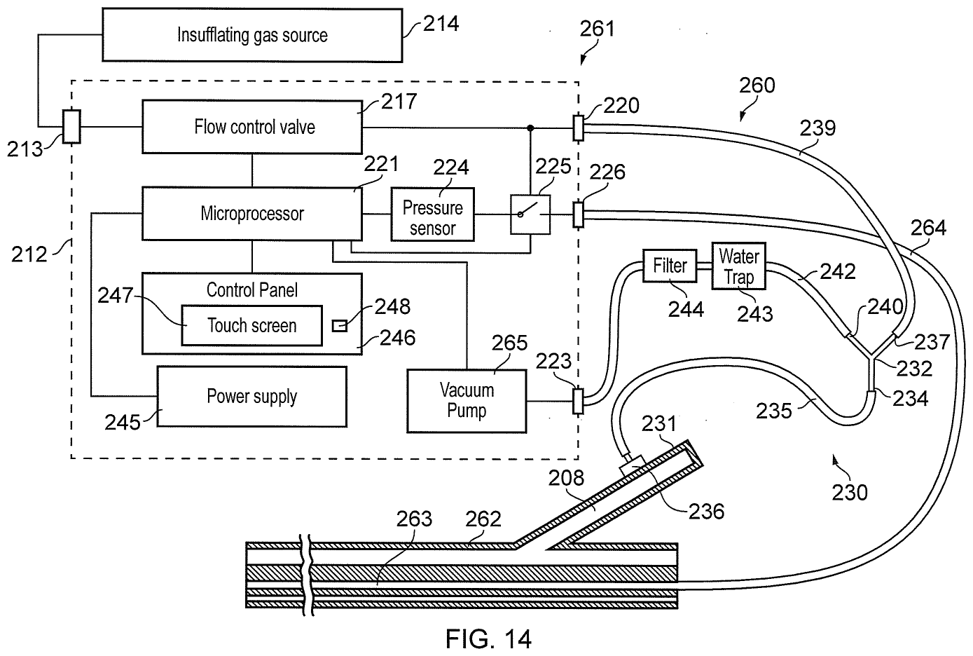

Referring now to FIG. 14 there is illustrated an insufflating system according to another embodiment of the invention indicated generally by the reference numeral 260. The insufflating system 260 is substantially similar to the insufflating system 201 and similar components are identified by the same reference numerals. The insufflating system 260 comprises an insufflator also according to the invention and indicated generally by the reference numeral 261 and an endoscope 262 connected to the insufflator 261 for supplying insufflating gas to a cavity to be insufflated in the body of a human or animal subject and for applying a vacuum thereto. The endoscope 262 is similar to the endoscope 205 of the insufflating system 201, and the instrument channel 208 thereof is connected to the insufflator 261 by a tube set 230, similar to the tube set 230 of the insufflating system 201, and is connected to the insufflator 261 by the tube set 230 in a similar manner as the endoscope 205 is connected to the insufflator 204 of the insufflating system 201. The only difference between the insufflating system 260 and the insufflating system 201 lies firstly in the insufflator 261, and secondly, in the manner in which the cavity pressure is monitored by the pressure sensor 224.

Dealing initially with the monitoring of the cavity pressure, in this case, due to the procedure being carried out in the cavity, an additional channel of the endoscope 262 is available for monitoring pressure in the cavity, namely, the channel 263 of the endoscope 262, which in this case is an insufflating channel 263 of the endoscope 262. Accordingly, the third port 226 is connected to the channel 263 of the endoscope 262 through a conduit 264, so that the pressure sensor 224 may continuously monitor the cavity pressure, and the microprocessor 221 is programmed to monitor the cavity pressure at millisecond intervals, typically at 10 millisecond intervals.

Turning now to the insufflator 261, in this embodiment of the invention the second control means of the insufflator 261 comprises a vacuum generating means, in this embodiment of the invention a vacuum pump 265 located in the housing 212 of the insufflator 261 and connected directly to the second outlet port 223. The vacuum pump 265 is operated under the control of the microprocessor 221 in response to the signal read from the pressure sensor 224 in a similar manner as the isolating valve 222 of the insufflator 204 is operated for applying vacuum to the second outlet port 223. Since the insufflator 261 comprises the vacuum pump 264, the second inlet port 215 may be dispensed with.

Accordingly, in this embodiment of the invention the microprocessor 221 when operating the insufflator 261 in the first and second operating modes, when a vacuum is to be applied to the cavity being insufflated, the microprocessor 221 operates the flow control valve 217 to isolate the first outlet port 220 from the insufflating source 214, as already described with reference to the insufflator 204, and operates the vacuum pump 265 to apply a vacuum to the second outlet port 223 and in turn to the cavity, instead of operating the isolating valve 222 from the isolating state to the communicating state.

However, since the pressure in the cavity is monitored continuously by the pressure sensor 224, and the microprocessor 221 monitors the cavity pressure at the 10 millisecond intervals, when operating in the first operating mode when the pressure in the cavity exceeds the predefined upper pressure, instead of operating the vacuum pump 265 for applying a vacuum to the cavity for a first predefined time period, once the vacuum pump has been operated to apply the vacuum to the cavity and the flow control valve 217 has been operated to isolate the cavity from the insufflating gas source 214, the microprocessor 221 reads the signal from the pressure sensor 224 at the 10 millisecond intervals, and on the signal read from the pressure sensor 224 being indicative of the cavity pressure having fallen to or below the set pressure, the microprocessor 221 deactivates the vacuum pump 265 and simultaneously operates the flow control valve 217 to reinstate insufflating gas to the cavity for maintaining the cavity at the set pressure.

Otherwise, the insufflating system 260 and its operation is similar to that of the insufflating system 201.

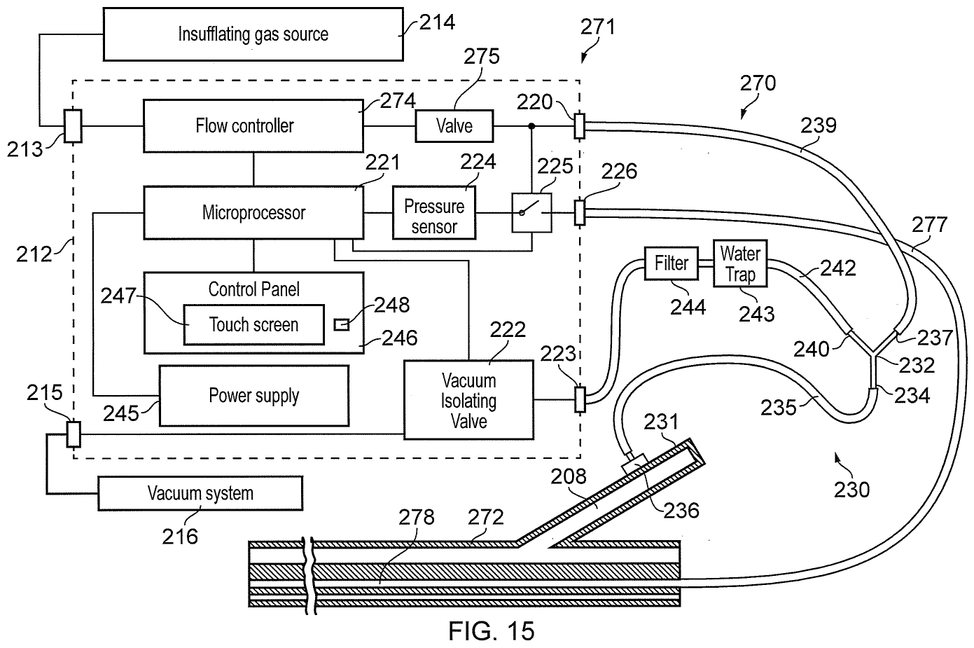

Referring now to FIG. 15, there is illustrated an insufflating system according to another embodiment of the invention indicated generally by the reference numeral 270 for insufflating a cavity in the body of a human or animal subject. The cavity may be the stomach of a subject, or any other cavity. The insufflating system 270 comprises an insufflator 271 and an endoscope 272. The insufflator 271 is substantially similar to the insufflator 204 described with reference to FIGS. 12 and 13, and similar components are identified by the same reference numerals. The insufflator 271 is operable in two operating modes, namely, a first operating mode and a second operating mode substantially similar to the two operating modes of the insufflator 204 of the insufflating system of FIGS. 12 and 13. The endoscope 272 is also substantially similar to the endoscope 205 of the insufflating system 201, described with reference to FIGS. 12 and 13, and similar components are also identified by the same reference numerals.

In the insufflator 271, instead of providing the first control means as a flow control valve, the first control means in the insufflator 271 comprises a flow controller 274 and a separate insufflating gas isolating valve 275. The flow controller 274 controls the flow rate of insufflating gas from the insufflating gas source 214 to the first outlet port 220, and in turn to the cavity being insufflated. The insufflating gas isolating valve 275 comprises a solenoid operated isolating valve selectively and alternately operable in an isolating state isolating the flow controller 274 from the first outlet port 220, and a communicating state communicating the flow controller 274 with the first outlet port 220 for accommodating insufflating gas from the flow controller 274 to the first outlet port 220.

The flow controller 274 is operated under the control of the microprocessor 221 in a substantially similar manner as the flow control valve 217 of the insufflator 204 is operated for controlling the flow rate of insufflating gas to the cavity. The insufflating gas isolating valve 275 is operated under the control of the microprocessor 221 between the isolating state to prevent the flow of insufflating gas from the flow controller to the first outlet port 220 and the communicating state to permit the supply of insufflating gas from the insufflating gas source 214 to the outlet port 220 in a substantially similar manner as the microprocessor 221 operates the flow control valve 217 to control the supply of insufflating gas from the insufflating gas source 214 to the outlet port 220 and in turn to the cavity being insufflated.

Accordingly, when insufflating gas is to be delivered to the cavity, the microprocessor 221 operates the insufflating gas isolating valve 275 into the communicating state, and when insufflating gas from the insufflating gas source 214 is to be isolated from the cavity being insufflated, the microprocessor 221 operates the insufflating gas isolating valve 275 into the isolating state to isolate the first outlet port 220 and in turn the cavity from the insufflating gas source 214.

Additionally, in this embodiment of the invention the pressure sensor 224 is connected to the third port 226 only, and cavity pressure is monitored by the pressure sensor 224 through the third port 226 and in turn through a conduit 277 connecting the second port 226 to one of the channels of the endoscope 272, other than the said instrument channel 208 thereof. For example, the pressure sensor 224 may be connected through the conduit 277 to an insufflating channel 278 or a vacuum channel if such were available in the endoscope 272.

In this embodiment of the invention the first outlet port 220 and the second outlet port 223 of the insufflator 271 are connected to the instrument channel 208 of the endoscope 272 adjacent the proximal end 231 of the endoscope 272 through the port 236 by a tube set 230 similar to the tube set 230 of the insufflating system described with reference to FIGS. 12 and 13 which connects the insufflator 204 to the endoscope 205 of the insufflating system 201, and in a similar manner as the tube set 230 connects the insufflator 204 to the endoscope 205.

The operation of the insufflating system 270, in general, is substantially similar to that of the operation of the insufflating system 201. However, in this embodiment of the invention since the pressure sensor 224 is continuously connected to the cavity being insufflated through the conduit 277 and the insufflating channel 278 of the endoscope 272, the microprocessor 221 is programmed to read the signal produced by the pressure sensor 224 indicative of the cavity pressure at the predefined time intervals of 10 milliseconds, and therefore, the operation of the insufflator 271 in the first and operating mode is similar to the operation of the insufflator 261 described with reference to FIG. 14 in the first operating mode. Operation of the insufflator 271 in the second operating mode is similar to the operation of the insufflator 204 described with reference to FIGS. 12 and 13 in the second operating mode.

Additionally, in this embodiment of the invention, if at any time while the vacuum isolating valve 222 is being operated in the communicating state for applying vacuum to the cavity, in either the first or second operating modes, the signal read from the pressure sensor 224 by the microprocessor 221 is indicative of the cavity pressure falling below a predefined lower pressure, the microprocessor 221 is programmed to operate the vacuum isolating valve 222 from the communicating state to the isolating state, and to simultaneously operate the insufflating gas isolating valve 275 from the isolating state to the communicating state and to operate the flow controller 274 to supply the insufflating gas at the flow rate at which insufflating gas had been delivered to the cavity prior to the insufflating gas isolating valve 275 being operated into the isolating state, or to increase the flow rate of the insufflating gas above that flow rate, in order to supply insufflating gas to the cavity to increase the cavity pressure to the set pressure.

Once the signal read from the pressure sensor 224 by the microprocessor 221 is indicative of the cavity pressure having returned to the set pressure, the microprocessor 221 operates the flow controller to reduce the flow rate of insufflating gas to the cavity in order to maintain the cavity pressure at the set pressure. However, if the cavity pressure continues to rise above the set pressure and reaches the predefined upper pressure, the microprocessor 221 is programmed to operate the insufflating gas isolating valve 275 from the communicating state to the isolating state and simultaneously to operate the vacuum isolating valve 222 from the isolating state to the communicating state until the cavity pressure has fallen to the set pressure.

In this embodiment of the invention the predefined lower pressure is approximately 6 mmHg below the set pressure. Although in some embodiments of the invention it is envisaged that the predefined lower pressure may lie in the range of 2 mmHg to 8 mmHg below the set pressure, while in other embodiments of the invention the predefined lower pressure may lie in the range of 2 mmHg to 10 mmHg below the set pressure.

Otherwise, the insufflator 271 and the insufflating system 270 and their operation are similar to that of the insufflator 204 and the insufflating system 201 described with reference to FIGS. 12 and 13.

While the predefined upper pressure has been described as being 1 mmHg above the set pressure, it is envisaged that the predefined upper pressure may be any upper safe pressure to which the cavity may be insufflated, and will largely be dependent on the nature of the cavity being insufflated. However, typically, in the case of a stomach, it is envisaged that the predefined upper pressure will lie in the range of 0.5 mmHg to 5 mmHg above the set pressure, and preferably, will lie in the range of 0.5 mmHg to 4 mmHg above the set pressure, provided of course that the set pressure is set at a pressure such that the difference between the set pressure and the predefined upper pressure does not result in the predefined upper pressure being of a dangerously high pressure value.

While the insufflators 204, 261 and 271 have been described when operating in the second mode to maintain the pressure in the cavity at the set pressure plus or minus 1 mmHg or 2 mmHg, in some embodiments of the invention it is envisaged that the pressure range within which the cavity pressure would be maintained during insufflating thereof in the second operating mode could be maintained at the set pressure plus or minus up to 3 mmHg.

While the means for switching the operation of the insufflators 204, 261 and 271 between the first and second operating modes has been described as comprising a membrane button operated switch, any other suitable means for switching the insufflators between the first and second operating modes may be provided. In some embodiments of the invention it is envisaged that the means for switching the insufflators between the first and second operating modes may comprise a foot pedal operated switch, which would be operated typically, by the surgeon or clinician. The foot pedal operated switch would be connected into the insufflator to the microprocessor.

While the insufflators 204, 261 and 271 have been described as forming a part of an insufflating system, the insufflators 204, 261 and 271, as well as any other insufflators according to the invention may be operated as standalone insufflators, and in which case, the insufflators may be connected to the cavity being insufflated by any suitable means, for example, through a colonoscope or a trocar, depending on the manner in which the procedure is being carried out.

While the first control means has been described as comprising a flow control valve, in some embodiments of the invention it is envisaged that the first control means may comprise a flow controller, which would not necessarily act to isolate the first outlet port from the insufflating gas source, and in which case, it is envisaged that as well as the flow controller, an isolating valve would be located between the flow controller and either the first outlet port or the first inlet port, and more commonly, would be located between the flow controller and the first outlet port.

While the insufflating systems 201, 260 and 270 have been described as comprising a bacterial-viral filter, any other suitable filter may be used, and while the filter has been described as being located in the third conduit, the filter may be located in any suitable location from the third outlet port of the Y-connector to and including the second inlet port 215. In some embodiments of the invention the filter may be located in the housing between the isolating valve or the vacuum pump and the second inlet or outlet ports.

While the insufflating systems 201, 260 and 270 have been described as comprising a water collecting means, in some embodiments of the invention the water collecting means may be omitted. It is also envisaged that other suitable water collecting means may be provided, besides a water trap. Additionally, where a water collecting means is provided, it will normally be located upstream of the filter in order to protect the filter from water entrained in the gases being evacuated from the cavity.

While the signal processor has been described as comprising a microprocessor, any other suitable signal processor may be used, for example, a microcontroller, a programmable logic controller or indeed any other suitable signal processor.

It will also of course be appreciated that any other suitable connecting means for connecting the insufflator to the endoscope may be used besides the tube set 230.

While the insufflating systems 201, 260 and 270 according to the invention and the insufflators according to the invention have been described for use in the carrying out of a procedure in the stomach of a subject, it will be readily apparent to those skilled in the art that the insufflating systems and the insufflators according to the invention may be used for carrying out any other procedures, for example, a procedure in the rectum, the colon, the intestine, the oesophagus, the abdominal cavity and in carrying out a procedure in the rectum or the colon where a colonoscope is being used, the colonoscope would be entered rectally into the subject. When the procedure is being carried out in the intestine of a subject, the entry of the endoscope or colonoscope into the subject would depend on the location in the intestine in which the procedure is being carried out. In the case of a procedure being carried out in the intestine closer to the stomach than the colon, it is envisaged that the endoscope would be entered orally into the subject, and otherwise, the colonoscope would be entered rectally into the subject.

While the set pressure has been described as being selectable in the range of 2 mmHg to 20 mmHg, it is envisaged that in some embodiments of the invention the set pressure may be selectable up to 25 mmHg. It will also be appreciated that while the set pressure has been described as being selectable, in some embodiments of the invention it is envisaged that the set pressure may be preset between 1 mmHg and 20 mmHg, and in some cases up to 25 mmHg.

While the second control means in the insufflators 204 and 271 have been described as comprising an isolating valve which is operable in two states only, namely, in an isolating state and a communicating state for controlling the supply of vacuum, it is envisaged that in some embodiments of the invention the second control means may comprise a vacuum pressure control valve, which would allow the pressure of the vacuum applied to the second outlet port to be varied and selectable. In which case, the microprocessor or other signal processor would be configured to control the vacuum pressure control valve in response to a signal from a switch means, for example, an external foot pedal operated switch configured to produce a signal to the microprocessor or other signal processor indicative of the vacuum pressure to be applied to the cavity. Typically, such a foot pedal or indeed a manually operated switch, may comprise a rheostat, so that the further the foot pedal is depressed, the greater would be the vacuum applied to the cavity, and in the case of a manually operated switch, for example, a button operated switch, the more the button operating the switch is depressed, the greater would be the vacuum applied to the cavity.

It is also envisaged that the microprocessor or other suitable signal processor may be configured to control the rate at which the vacuum pump applies the vacuum to the cavity, for in turn varying the vacuum applied to the cavity. In which case, it is envisaged that the microprocessor or signal processor would be configured to be responsive to a signal from a suitable switch means, for example, a foot pedal operated switch or a manually operated button switch, such as a rheostat switch, so that the more the pedal is depressed or the button of the button operated switch is depressed, the greater would be the vacuum applied to the cavity by the vacuum pump.

While in the embodiments of the invention described with reference to FIGS. 12 to 14, a valve 225 has been provided operable under the control of the microprocessor 221 for selectively switching the pressure sensor 224 between the third port 226 and the first outlet port 220, in some embodiments of the invention it is envisaged that the valve 225 may be omitted, and in which case, two pressure sensors may be provided, one pressure sensor being connected to the third port 226 and the other pressure sensor being connected to the first outlet port 220. The microprocessor 221 would be programmed to read the signals from the appropriate one of the pressure sensors. If the third port 226 is not connected to the cavity being insufflated through the endoscope or otherwise, the microprocessor would read the signal from the pressure sensor connected to the first outlet port 220, and if the third port 226 is connected for communicating with the cavity being insufflated, the microprocessor would read the signal from the pressure sensor connected to the third port 226. It is also envisaged that the pressure sensor may be connected to the second outlet port 223 and could also monitor the cavity pressure through the second outlet port.

It is also envisaged that three separate pressure sensors may be provided, one of which would communicate with the first outlet port 220, the other of which would communicate with the second outlet port 223, and the third of which would communicate with the third port 226, and the microprocessor would be configured to read the signal from the appropriate one of the three pressure sensors to determine the cavity pressure.

It will also be appreciated that a switch other than a membrane switch may be provided for selecting the operating mode of the insufflators described with reference to FIGS. 12 to 15.

It is also envisaged that in some embodiments of the invention described with reference to FIGS. 12 to 15 a means for selectively connecting the tube set between the instrument channel and a channel other than the instrument channel, for example, an insufflating channel or a vacuum channel of the endoscope may be provided. In which case, it is envisaged that the tube set would be connected through a spool valve or other suitable valve to both the instrument channel and the other one of the channels of the endoscope, and the spool valve or such other suitable valve would typically be operated by a foot pedal operated switch which would allow the tube set to be selectively and alternately connected to the instrument channel or the other one of the channels of the endoscope, such as the insufflating channel or the vacuum channel as the case may be.

While the sleeve gastroplasty procedure has been described using the insufflating system 1 including the insufflator 3 and the endoscope 5 of FIGS. 1 to 3, 5 and 8 all according to the invention, it is envisaged that in some embodiments of the invention the insufflator 3 may be used also for carrying out a sleeve gastroplasty procedure in the stomach of a subject without the endoscope 5, but rather, with a conventional endoscope, whereby insufflating of the stomach would be carried out solely through the insufflating channel, and the pressure in the stomach could be monitored either through the instrument channel or through the insufflating channel, with insufflating being paused to allow monitoring of the pressure in the stomach through the insufflating channel.

While the endoscopes of FIGS. 1 to 3, 5 and 8 have been described as comprising an instrument sensor, while this is desirable, in some cases the instrument sensor may be omitted, and the operating of the output valve 47 and the input valve 57 may be operated by other means, for example, manually or in response to an input signal to the microcontroller of the insufflator produced, for example, by another foot or hand operated switch. Alternatively, in some embodiments of the invention where a solenoid or other motorised valve is provided for switching insufflating gas from the instrument channel to the insufflating channel and vice-versa, the valve may be located on the endoscope adjacent the proximal end thereof and the valve may be manually operated or otherwise operated or be locatable in the insufflator.

It will also be appreciated that where it is desired to switch the pressure sensor from the insufflating channel to the instrument channel and vice-versa, this may be done manually by a suitable valve either located in the insufflator or on the endoscope typically, adjacent the proximal end of the endoscope. Such a valve may be manually or otherwise operable. Indeed, in some embodiments of the invention the input valve 57 may be dispensed with as discussed above.

While the insufflating system of FIG. 1 has been described as comprising the insufflator according to the invention and the endoscope according to the invention, in some embodiments of the invention it is envisaged that the insufflating system may be provided with the insufflator according to the invention and with a conventional endoscope. It is also envisaged in some embodiments of the invention that the insufflating system may be provided with the endoscope according to the invention and a conventional insufflator.

It is also envisaged that the endoscope described with reference to FIGS. 1 to 3, 5 and 8 may be provided with an insufflator incorporated into the endoscope, and the insufflator may or may not be configured to control the pressure at which the insufflating gas is supplied at two pressures, which may be selectable or otherwise. In other words, in some cases the insufflator incorporated into the endoscope may be configured to regulate the insufflating gas at one pressure only which may or may not be selectable.

In some embodiments of the invention described with reference to FIGS. 1 to 11 where an instrument is inserted in the instrument channel, and can be reasonably well sealed at the proximal end of the instrument channel proximal of the branch channel 18, and where the instrument is of a size which would permit insufflating of the cavity through the instrument channel, it is envisaged that as well as delivering the insufflating gas to the cavity through the insufflating channel, insufflating gas may also be delivered to the cavity through the instrument channel even with or without the instrument in the instrument channel. In this case, appropriate valving would be provided for connecting the insufflator to both the instrument channel and the insufflating channel simultaneously in the endoscope, and during periods while the insufflating gas is being delivered through the insufflating channel, the delivery of the insufflating gas through either the insufflating channel or the instrument channel would be intermittently interrupted to allow the pressure in the cavity to be monitored through the relevant one of the insufflating channel or the instrument channel.

It is also envisaged that in some embodiments of the invention as well as or instead of delivering the insufflating gas through the insufflating channel when an instrument is located in the instrument channel, insufflating gas may also, or instead, be delivered through the vacuum channel when the vacuum channel is not required to suction from or apply a vacuum to the cavity being insufflated. In which case appropriate valving would be provided between the insufflator and the endoscope.

It is also envisaged that in some embodiments of the invention the supply of the pressurised water may be provided from the insufflator which would be provided with a pressurised water supply, and the control of the delivery of the pressurised water supply, whether it is to be delivered through the instrument channel or the insufflating channel would be controlled by a valving system in the insufflator. Alternatively, the valving system may be located in the endoscope, and in which case, on receiving a signal from the endoscope that a supply of water is required, the insufflator would provide the supply of water to the endoscope, which through the valving system in the endoscope, would be applied to the appropriate one of the insufflating channel and the instrument channel. In which case, it is envisaged that a surgeon or clinician would merely have to press a single button, or issue a single voice command in order to have the pressurised water supply delivered to the appropriate one of the instrument channel and the insufflating channel, which would typically be determined by the insufflator.

It is also envisaged that in some embodiments of the invention a back pressure monitor may be provided for monitoring the back pressure adjacent the proximal end of the instrument channel when the insufflating gas is being delivered through the instrument channel, and signals indicative of the monitored back pressure value would be read by the microcontroller. In the event of the back pressure exceeding a predefined back pressure value which would indicate that sufficient insufflating gas is not being delivered to the cavity through the instrument channel, for example, as a result of an instrument therein, the microcontroller would operate the output valve to switch the insufflating gas from the instrument channel to the insufflating channel so that insufflating of the cavity would continue through the insufflating channel. In which case, the cavity pressure would be monitored through the insufflating channel by intermittently pausing the delivery of the insufflating gas to the insufflating channel in order to allow the cavity pressure to be monitored through the insufflating channel. It is envisaged that the microcontroller of the insufflator would be programmed to monitor the back pressure monitor during periods when the insufflating gas is being delivered to the cavity through the instrument channel, and also when it is known that an instrument is located in the instrument channel. It is also envisaged that in some cases, even when the back pressure monitored by the back pressure monitor exceeds the predefined back pressure value, the microcontroller may operate the insufflator to continue insufflating of the cavity through the instrument channel, and also through the insufflating channel. This however would require additional valving which would be provided either in the insufflator or in the endoscope.

It is also envisaged that in some embodiments of the invention described with reference to FIGS. 1 to 11 instead of providing a plurality of ports tapped into the branch channel 18 for connecting the first output gas line 52, the first input gas line 62, the vacuum line 68 and the pressurised water supply to the branch channel 18, it is envisaged that a single manifold may be tapped into the branch channel 18, and the first output gas line 52, the first input gas line 62, the vacuum line 68 and the pressurised water supply would be connected to the manifold, which in turn would be tapped into the branch channel 18.

While the insufflator described with reference to FIG. 1 has been described in conjunction with an insufflating system which also comprises the endoscope, it is envisaged that the insufflator according to the invention may be provided as a standalone insufflator, and in which case the output valve 47 and the input valve 57 may be omitted, and the output valve would be replaced by a single output port through which insufflating gas would be delivered from the insufflator, and the input valve would be replaced by an input port through which the pressure in the cavity would be monitored by the pressure sensor. It will also be appreciated that whether the insufflator is being provided as a stand along unit or in conjunction with an endoscope in an insufflating system, the vacuum control valve may be omitted, since the vacuum would only be applied when the vacuum pump is operated.

It will also be appreciated that in some embodiments of the invention the insufflator may be provided without a vacuum pump, and in which case, the insufflator would be adapted for connecting to a vacuum system of a hospital. In such cases where the insufflator is adapted for connecting to the vacuum system of a hospital, the application of the vacuum to the cavity would be controlled by the vacuum control valve, which would be operated under the control of the microcontroller.

While the insufflating system, the insufflator and the endoscope have been described for use in carrying out a procedure in the stomach of a subject, it will be readily apparent to those skilled in the art that the insufflating system, the insufflator and the endoscope may be used for carrying out a procedure in any cavity, lumen or vessel.

1. An insufflator for insufflating a cavity in the body of a human or animal subject, the insufflator comprising:
a first control means for controlling the supply of insufflating gas to the cavity,
a second control means for controlling application of a vacuum to the cavity,
a pressure monitoring means for monitoring pressure in the cavity (cavity pressure) and for producing a signal indicative of the cavity pressure, and
a signal processor configured to read the signal from the pressure monitoring means and to control the first and second controls means in response to the signal read from the pressure monitoring means, wherein the signal processor is configured to selectively operate the insufflator in selectable ones of a first operating mode and a second operating mode, and
in the first operating mode the signal processor is configured
to operate the first control means in response to the signal read from the pressure monitoring means to control the supply of insufflating gas to the cavity for maintaining the cavity pressure substantially at a set pressure, and
on the signal read from the pressure monitoring means being indicative of the cavity pressure exceeding a predefined upper pressure greater than the set pressure, the signal processor is configured
to operate the second control means to apply a vacuum to the cavity until the cavity pressure falls to the set pressure or
to apply a vacuum to the cavity for a first predefined time period, and
in the second operating mode, the signal processor is configured
to control the first and second control means to sequentially and alternately supply insufflating gas to the cavity and to apply a vacuum to the cavity in a plurality of sequential pressure/vacuum cycles.

2. An insufflator as claimed in claim 1 in which the signal processor is responsive to failure of the cavity pressure falling to or below the set pressure at the end of the first predefined time period to operate the second control means to apply the vacuum to the cavity for at least one second time period, and preferably, the signal processor is responsive to failure of the cavity pressure falling to or below the set pressure at the end of each second time period to operate the second control means to apply the vacuum to the cavity for another second time period.

3. An insufflator as claimed in claim 2 in which the signal processor is programmed to compute the time duration of the first one of the second time periods and each subsequent second time period as a function of the difference between the set pressure and the cavity pressure at the end of the immediately preceding time period, and/or as a function of the rate at which the cavity pressure dropped during the immediately preceding time period.

4. An insufflator as claimed in claim 1 in which in the second operating mode, the signal processor is programmed to operate the first and second control means during each pressure/vacuum cycle to maintain the cavity pressure within a predefined pressure range about the set pressure, and preferably, the predefined pressure range about the set pressure lies in the range of 3 mmHg above the set pressure and 3 mmHg below the set pressure.

5. An insufflator as claimed in claim 1 in which in the second operating mode, the signal processor is programmed to operate the first control means to supply the insufflating gas to the cavity for a predefined gas supply time period in each pressure/vacuum cycle, preferably, lying in the range of 4 seconds to 10 seconds, and to operate the second control means to apply vacuum to the cavity for a predefined vacuum application time period in each pressure/vacuum cycle, preferably, lying in the range of 1 second to 5 seconds.

6. An insufflator as claimed in claim 5 in which the duration of the predefined vacuum application time period lies in the range of one eighth to one half of the duration of each pressure/vacuum cycle.

7. An insufflating system for insufflating a cavity in the body of a human or animal subject, the insufflating system comprising:
the insufflator as claimed in claim 1,
an endoscope comprising a channel capable of accommodating an insufflating gas to the cavity of the subject, and
a connecting means connecting the insufflator to the said channel for alternately applying the insufflating gas and the vacuum to the said channel of the endoscope.

8. An insufflating system as claimed in claim 7 in which the connecting means is adapted for connecting to the first control means and to the second control means and to the said channel of the endoscope.

9. An insufflating system as claimed in claim 7 in which the pressure monitoring means is connected to the said channel of the endoscope, or to a channel of the endoscope other than the said channel thereof.

10. An insufflating system as claimed in claim 7 in which the said channel of the endoscope capable of accommodating insufflating gas to the cavity of a subject comprises one of an instrument channel of the endoscope, an insufflating gas channel of the endoscope, a vacuum channel of the endoscope, or a channel extending through a tubular member attached to the endoscope and extending externally along the endoscope.

11. An insufflating system for insufflating a cavity in the body of a human or animal subject, the insufflating system comprising:
an insufflator comprising a first control means for controlling the supply of insufflating gas to a cavity in the body of a subject for insufflating the cavity to a set pressure, and a second control means for applying a vacuum to the cavity,
an endoscope comprising a channel capable of accommodating an insufflating gas to the cavity of the subject, and
a connecting means connecting the insufflator to the said channel of the endoscope for alternately applying insufflating gas and a vacuum to the cavity through the said channel of the endoscope.

12. An insufflating system as claimed in claim 11 in which the first control means and the second control means are configured for sequentially supplying insufflating gas and applying the vacuum to the said channel of the endoscope in sequential pressure/vacuum cycles.

13. An insufflating system as claimed in claim 12 in which the first control means is configured for supplying insufflating gas to the said channel of the endoscope during each pressure/vacuum cycle for a predefined gas supply time period, and the second control means is configured for applying the vacuum to the said channel of the endoscope during each pressure/vacuum cycle for a predefined vacuum application time period, and preferably, each predefined gas supply time period lies in the range of 4 seconds to 10 seconds, and preferably, each predefined vacuum application time period lies in the range of 1 second to 5 seconds, and advantageously, the duration of each predefined vacuum application time period lies in the range of one eighth to one half of the duration of one insufflating gas/vacuum cycle.

14. An insufflating system as claimed in claim 13 in which the second control means is responsive to the cavity pressure exceeding the set pressure or a predefined upper pressure greater than the set pressure for applying the vacuum to the said channel of the endoscope, and preferably, for applying the vacuum to the said channel of the endoscope for one predefined vacuum application time period, and preferably, for applying the vacuum to the said channel of the endoscope until the cavity pressure is reduced to or below the set pressure.

15. A method for insufflating a cavity in the body of a human or animal subject with an insufflator operable in selectable first and second operating modes, the method comprising:
monitoring the pressure in the cavity (cavity pressure),
operating the insufflator in the first operating mode for insufflating the cavity to maintain the cavity pressure at a set pressure, and
in response to the cavity pressure exceeding the set pressure or a predefined upper pressure greater than the set pressure operating the insufflator to apply a vacuum to the cavity until the cavity pressure falls to the set pressure, or
operating the insufflator to apply a vacuum to the cavity for a first predefined time period, and
operating the insufflator in the second operating mode for sequentially and alternately supplying insufflating gas to the cavity and applying a vacuum to the cavity in a plurality of sequential pressure/vacuum cycles.

16. A method as claimed in claim 15 in which on failure of the cavity pressure to fall to or below the set pressure at the end of the first predefined time period, the insufflator is operated to apply the vacuum to the cavity for at least one second time period, and preferably, on failure of the cavity pressure to fall to or below the set pressure at the end of each second time period the insufflator is operated to apply the vacuum to the cavity for another second time period.

17. A method as claimed in claim 16 in which the time duration of the first one of the second time periods and each subsequent second time period is computed as a function of the difference between the set pressure and the cavity pressure at the end of the immediately preceding time period, and/or as a function of the rate at which the cavity pressure dropped during the immediately preceding time period.

18. A method as claimed in claim 16 in which the time duration of each one of the second time periods is the same as the time duration of the first predefined time period.

19. A method as claimed in claim 15 in which in the second operating mode, the cavity pressure is maintained within a predefined pressure range about the set pressure during each pressure/vacuum cycle, and preferably, the predefined pressure range about the set pressure lies in the range of 3 mmHg above the set pressure and 3 mmHg below the set pressure.

20. A method as claimed in claim 15 in which insufflating gas is supplied to the cavity during each pressure/vacuum cycle for a predefined gas supply time period, preferably, lying in the range of 4 seconds to 10 seconds, and vacuum is applied to the cavity during each pressure/vacuum cycle for a predefined vacuum application time period, preferably, lying in the range of 1 second to 5 seconds.

21. A method as claimed in claim 20 in which the duration of the predefined vacuum application time period lies in the range of one eighth to one half of the duration of each pressure/vacuum cycle.

22. A method as claimed in claim 15 in which insufflating gas is supplied to the cavity and the vacuum is applied to the cavity from the insufflator through a channel in an endoscope capable of accommodating insufflating gas to the cavity.

23. A method as claimed in claim 22 in which the said channel of the endoscope is connected through a connecting means to a first control means of the insufflator which controls the supply of insufflating gas to the cavity for accommodating insufflating gas to the cavity, and to a second control means of the insufflator which controls the application of the vacuum to the cavity for applying the vacuum to the cavity.

24. A method as claimed in claim 22 in which the said channel of the endoscope capable of accommodating insufflating gas to the cavity of a subject comprises one of an instrument channel of the endoscope, an insufflating gas channel of the endoscope, a vacuum channel of the endoscope, or a channel extending through a tubular member attached to the endoscope and extending externally along the endoscope.

25. A method as claimed in claim 22 in which the cavity pressure is monitored through the said channel of the endoscope, or through a channel of the endoscope other than the said channel.

26. A method for insufflating a cavity in the body of a human or animal subject comprising:
sequentially and alternately supplying insufflating gas to the cavity and applying a vacuum to the cavity in a plurality of sequential pressure/vacuum cycles for maintaining an intermittent flow of insufflating gas through the cavity to withdraw smoke and/or other undesirable gases from the cavity.

27. A method for insufflating a cavity in the body of a human or animal subject and for controlling the pressure at which the cavity is insufflated substantially at a set pressure, the method comprising:
inserting an endoscope into the cavity,
connecting a channel of the endoscope capable of accommodating an insufflating gas to the cavity to an insufflator, and
sequentially and alternately applying insufflating gas to the said channel of the endoscope for delivery to the cavity and applying a vacuum to the said channel of the endoscope in sequential pressure/vacuum cycles for withdrawing the smoke or the gas from the cavity with the cavity maintained substantially at the set pressure.

28. An insufflator for insufflating a cavity in the body of a human or animal subject, the insufflator comprising:
a first control means for controlling the supply of insufflating gas to the cavity,
a second control means for controlling application of a vacuum to the cavity, and
a signal processor programmed to sequentially and alternately operate the first control means to supply insufflating gas to the cavity, and the second control means to apply a vacuum to the cavity in a plurality of sequential pressure/vacuum cycles for maintaining an intermittent flow of insufflating gas through the cavity to withdraw smoke and/or other undesirable gases from the cavity.
